(19) United States
(12) Patent Application Publication
(10) Pub. No.: US 2003/0124585 A1
(43) Pub. Date: Jul. 3, 2003

(54) Type II gonadotropin-releasing hormone receptor and polynucleotides encoding therefor

(75) Inventors: Robert Peter Millar, North Berwick (GB); Steven Lowe, Cape Town (ZA); Darrell Conklin, Seattle, WA (US)

Correspondence Address: DRINKER BIDDLE & REATH, ONE LOGAN SQUARE, 18TH AND CHERRY STREETS, PHILADELPHIA, PA, 19103-6996, US

(73) Assignee: Medical Research Council, London (GB)

(21) Appl. No.: 10/263,872

(22) Filed: Oct. 2, 2002

Related U.S. Application Data
(63) Continuation-in-part of application No. PCT/GB01/01755, filed on Apr. 17, 2001.
(60) Provisional application No. 60/215,232, filed on Jun. 30, 2000.

(30) Foreign Application Priority Data
Apr. 15, 2000 (GB) 0009269.2
Jun. 17, 2000 (GB) 0014761.1

(51) Int. Cl. C12Q 1/68; C07H 21/04; C07K 14/72; C12P 21/02; C12N 5/06

(52) U.S. Cl. 435/6; 435/69.1; 435/320.1; 435/325; 530/350; 536/23.5

(57) ABSTRACT

There is provided polynucleotides encoding the full sequence for the marmoset and human Type II gonadotropin-releasing hormone receptors (Type II GnRH-R). The corresponding amino acid sequences are also provided.

### CROSS REFERENCE TO RELATED APPLICATION

This is a continuation-in-part of International Application No. PCT/GB01/01755, filed Apr. 17, 2001, which in turn claimed priority of U.S. provisional No. 60/215,232 filed Jun. 30, 2000, the entire disclosure of which is incorporated herein by reference.

### TECHNICAL FIELD

The present invention relates to a novel Type II gonadotropin-releasing hormone receptor (Type II GnRH-R), to genetically engineered host cells able to express the Type II GnRH-R, and the ligands and antibodies therefor.

### BACKGROUND OF THE INVENTION

Type I gonadotropin-releasing hormone (GnRH) is a decapeptide released from the hypothalamus, and acts through.receptors to regulate the secretion of gonadotropins required for reproductive function (see Fink et al., “Gonadotrophin secretion and its control”, The Physiology of Reproduction, E Knobil and I Neill, New York, Raven Press, pages 1349-1377, 1988).

Receptors for Type I GnRH (ie Type I GnRH-R) are members of the large G-protein-coupled receptor family and are preferentially coupled to phosphoinositidase C via the G_{q}/G₁₁ family of G proteins. Typically Type I GnRH-Rs are located in the gonadotroph cells of the anterior pituitary gland (where binding of Type I GnRH leads to release of the gonadotropins luteinising hormone and follicle-stimulating hormone), as well as on the central and peripheral nervous systems, gonads, placenta and on certain tumours, such as breast and prostate. Type I GnRH receptors may display both up and down regulation and Type I GnRH agonists have been used in management of prostate and breast cancer, as well as to stimulate gonadotropin secretion in the treatment of infertility.

Expression of mouse and rat Type I GnRH-R was first achieved by injecting poly(A)+mRNA from a suitable source (eg from the pituitary gland) into Xenopus oocytes (see, for example, Eidne et al., J. Mol. Endocr. Vol 1, pages R9-R12, 1988; Yoshida et al., Molecular Endocrinology, Vol 3, pages 1953-1960, 1989; and Sealfon et al., Molecular Endocrinology, Vol 4, pages 119-124, 1990). This system allowed some characterisation of the pharmacology of the Type I GnRH-R.

The protein-encoding nucleotide sequence of the murine Type I GnRH-R was first published by Tsutsumi et al., (Molecular Endocrinology, Vol 6, pages 1163-1169, 1992) together with the deduced amino acid sequence for murine Type I GnRH-R. Eleven different forms of GnRH in vertebrates have been identified to date (see King and Millar, “Co-ordinated evolution of GnRHs and their receptors”, in GnRH Neurons: Gene to Behavior, Eds. I. S. Parhar and Y. Sakuma, Brain Shuppan, Tokyo, pages 51-77, 1997; see Sealfon et al., Endocr. Rev. 18:180-205, 1997; Millar et al., “Plasticity in the structural and functional evolution of GnRH: A peptide for all seasons”, in Proceedings of the XIIIth International Conference of Comparative Endocrinology, Eds. S. Kawashima and S. Kikuyama, Moduzzi Editore, Italy, pages 15-27, 1997; and see Sherwood et al., General and Comparative Endocrinology, 112, 1998). Many of these different GnRH forms are in fact variants of GnRH Type I. However, distinct Type II and Type III GnRHs have been identified.

Type II GnRH was originally isolated from chicken brain (see Millar and King, News Physiological Science, 3:49-53, 1988) and was initially termed “chicken GnRH II” or “cGnRH II”. Subsequent investigations have revealed that this isoform is present in most vertebrate species, and of all the GnRH isoforms GnRH II is the most ubiquitous. The wide distribution of GnRH II suggests on important function and it is postulated to have a neuromodulatory, and possibly a neuroendocrine, role in the central and peripheral nervous systems (see Millar and King, 1988, supra). GnRH II has been shown to regulate M currents (K⁺ channels) in the sympathetic ganglion (Bosma et al., in G proteins and Signal Transduction, The RockeFeller University Press, pages 43-59, 1990) and stimulates reproductive behaviour (see King et al., in GnRH Neurones:Gene to Behavior, eds. Parhar (Brain Shuppan), Tokyo, pages 51-77, 1997. It has also been postulated that GnRH II acts as a specific FSH-releasing agent (Millar et al. Ref No. 33).

Type II GnRH is highly expressed in kidney, bone marrow and prostate tissues as well as the extrahypothalamic brain (White et al. Ref No. 15).

To date only partial sequence information has been available for a Type II GnRH-R. Specifically, Millar et al. (in Journal of Endocrinology, 162:117-126, 1999) reported a continuous nucleotide sequence of 1642 nucleotides of the human gene, obtained by screening the human genome EST (expressed sequence tag) database. The EST sequences were confirmed in the cloned human gene and in PCR products of cDNA from several tissues. All the EST transcripts detected were in the antisense orientation with respect to the novel GnRH receptor sequences herein described and were highly expressed in a wide range of human brain and peripheral tissues.

PCR analysis of the cDNA partial sequence obtained by Millar et al., revealed that an intronic sequence equivalent to intron 2 of human Type I GnRH-R was retained. The intron itself was not spliced out in the transcript, but this was expected for anti-sense transcripts, as candidate donor and acceptor sites were only present in the gene when transcribed in the orientation encoding the GnRH receptor homolog. Despite extensive 5′ RACE studies Millar et al. did not obtain any transcript 5′ to the sequence corresponding to intron 2 of human Type I GnRH-R, and the antisense transcripts terminated in poly A due to the presence of a polyadenylation signal sequence in the putative intron 2 when transcribed in the antisense orientation (see FIG. 1).

None of the sequence revealed any contiguous open-reading frame which would translate a functional protein. Millar et al. concluded that the putative receptor was probably a pseudogene representing a receptor which had become redundant and further investigations have revealed that the full-length antisense transcript encodes a novel ribonucleoprotein (RNP) which localised to chromosome 14 (Ref 26). Subsequently it was found that the gene was a pseudogene for both RNP and Type II GnRH-R. The presence of the RNP explains the widespread tissue expression observed and it is significant that only the 3′ untranslated sequences of the RNP cDNA overlap the putative GnRH Type II receptor sequences encoding the equivalent of exon 1 and exon 2.

Despite the findings of Millar et al. which suggest that an operative version of GnRH-R Type II does not exist in mammals, we have now obtained the full nucleotide sequence encoding the Type II GnRH-R from marmoset and also complete nucleotide sequences (including exon I) encoding the human Type II GnRH-R. The human Type II GnRH-R herein reported has been localised to chromosome 1 (1q12-21).

### BRIEF SUMMARY OF THE INVENTION

The present invention thus provides a polynucleotide encoding a functional Type II gonadotropin-releasing hormone receptor (Type II GnRH-R) peptide.

Alternative coding sequences for the human Type II GnRH-R sequences are provided, which encode variants of the Type II GnRH-R. The first human Type II GnRH-R sequence (SEQ ID No. 3) provides one a sequence encoding Type II GnRH-R. The second human Type II GnRH-R sequence (SEQ ID No. 36) provides three potential variants of this sequence. These sequence variations may be the result of allelic polymorphisms or other causes. More specifically, SEQ ID No. 36 has four nucleotides which vary from SEQ ID No. 3; i.e. position 481(G or C), 897(either C or T), 1069(A→T) and 1073(A→G). Thus the three possible variants provided by SEQ ID No. 36 are where the positions are as follows:

Variant 1—481=C, 897=T, 1069=T and 1073=G

Variant 2—481=G, 897=T, 1069=T and 1073=G

Variant 3—481=C, 897=C, 1069=T and 1073=G

A prefered variant is variant 1.

The term “peptide” is used herein to refer to any peptidal compound without connotation of size, and includes therefore larger molecules which elsewhere may alternatively be termed “polypeptides” or “proteins” also fall within this definition.

In one embodiment the peptide encoded comprises at least a portion of exon I. By “exon I” we refer to that portion of Type II GnRH-R which corresponds to and exhibits substantial homology with exon I of Type I GnRH-R. In particular we refer to a peptide which includes over 90% of the amino acid sequence 1 to 170 in the marmoset Type II GnRH-R sequence of SEQ ID No. 2 or to a peptide which includes the equivalent amino acids of the human Type II GnRH-R sequences. Thus, for example, exon I refers to amino acid nos. 1 to 168 of splicing alternative 1 as set out in SEQ ID No. 5 (see FIG. 3). The above terminology has been adopted for clarity; the short splice referred to below is herein designated intron 1′ to distinguish it from intron 1. Consequently the short exon (exon 1′) comprising amino acids nos. 1 to 9 of the human Type II GnRH-R peptides is incorporated into exon I as herein defined.

Preferred embodiments of the present invention include polynucleotides having a nucleotide sequence as set out in SEQ ID No. 1 (marmoset) or SEQ ID No. 3 or 36 (human) or polynucleotides which encode a polypeptide having an amino acid sequence as set out in SEQ ID No. 2 (marmoset) or SEQ ID Nos. 4, 6, 8, 10, 37, 39, 41 or 43 (human).

As shown in FIG. 3 the marmoset amino acid sequence of Type II GnRH-R has over 90% homology with the corresponding human sequences. Accordingly, the present invention incorporates any polypeptide having at least 90% homology with the amino acid sequence of SEQ ID Nos. 1, 3 or 36.

When the human nucleotide sequences are compared to that of the marmoset, there is an apparent single-base deletion at position 30 of the coding sequence. The earlier EST sequence (Genbank BG036291) matches the 5′ end of the sequence, but the match ends at nucleotide 29 of the coding sequence and continues from nucleotide 290 of the coding sequence, indicating that this is a splice site. In the chicken receptor an intron was also located in a similar position. Excision of a very short intron (intron 1′) at this position would account for the frame shift; very short introns have been noted in other genes, for example the mouse alpha-7 integrin gene has an intron of only 16 bases (Genbank L23422). There are three alternative splicing possibilities, involving 5-, 8- or 38-base deletions, each of which would restore the open reading frame of the human Type II GnRH-R.

The start of the human Type II GnRH-R amino acid sequence following each of the three splicing alternatives is set out below:

Splicing alternative 1:
(5 bases deleted from position 29 on)

| | | |
|---|---|---|
| | Met Ser Ala Gly Asn Gly Thr Pro Trp | 9 |
| 1 | ATG TCT GCA GGC AAC GGC ACC CCT TGG | |
| | Ala Ala Gly Glu Glu Val Trp Ala | 17 |
| 28 | GCA GCG GGG GAG GAG GTC TGG GCT | |

Splicing alternative 2:
(8 bases deleted from position 29 on)

| | | |
|---|---|---|
| | Met Ser Ala Gly Asn Gly Thr Pro Trp | 9 |
| 1 | ATG TCT GCA GGC AAC GGC ACC CCT TGG | |
| | Ala Gly Glu Glu Val Trp Ala Gly | 17 |
| 28 | GCG GGG GAG GAG GTC TGG GCT GGA | |

Splicing atlernative 3:
(38 bases deleted from position 29 on)

| | | |
|---|---|---|
| | Met Ser ALa Gly Asn Gly Thr Pro Trp | 9 |
| 1 | ATG TCT GCA GGC AAC GGC ACC CCT TGG | |
| | Val Glu Val Glu Gly Ser Glu Leu | 17 |
| 28 | GTG GAG GTG GAG GGC TCA GAG CTG | |

The full sequences for human Type II GnRH-Rs according to the first splicing alternative are set out in SEQ ID Nos. 5 and 6, 38 and 39; according to the second splicing alternative in SEQ ID Nos. 7 and 8, 40 and 41; and according to the third splicing alternative in SEQ ID Nos. 9 and 10, 42 and 43.

As shown in SEQ ID Nos. 3 and 36, the human nucleotide sequences includes an apparent stop codon in the first part of exon 2. In the marmoset the equivalent codon (shown at nucleotides 449-452 of SEQ ID No. 1) represents an arginine. A modified version of SEQ ID No. 3 or 36 whereby the stop codon is engineered to represent an amino acid (for example arginine) is hereby incorporated. However, we believe that the codon TGA does not function as stop codon, but is instead translated as selenocysteine. In eukaryotes decoding of TGA as selenocysteine requires the selenocysteine insertion element (SECIS) in the 3′ UTR of the mRNA (see Copeland et al., EMBO J, 19(2): 306-14, 2000; and Fagegaltier et al., Nucleic Acids Research, 29(14): 2679-80, 1995). The putative UTR of the sequence (see SEQ ID Nos. 3 and 36) was found to contain the SECIS pattern RTGAN{13,15}AARN{23,26}GA. Thus, selenocysteine insertion at that position of the protein (amino acid no 177 in SEQ ID Nos. 6 and 39; amino acid no 176 in SEQ ID Nos. 8 and 41; amino acid No. 166 in SEQ ID Nos. 10 and 43) is possible. In SEQ ID Nos. 3, 5, 7, 9, 36, 38, 40 and 42 the TGA in shown as Xaa (and defined as TGA in the preamble to the sequence) simply to maintain the coding sequence as a whole in the PATENTIN program.

Desirably the polynucleotide according to the present invention encodes a peptide which is able to bind specifically to Type II GnRH and, preferably, is able to function as a receptor therefor.

Those skilled in the art will appreciate that the nucleotide sequences of SEQ ID Nos. 1, 3, 5, 7, 9, 36, 38, 40 and 42 correspond to particular alleles of the marmoset and human gene respectively, and that allelic variation is likely. Allelic variants can be cloned by probing cDNA or genomic libraries from different individuals according to standard procedures. Allelic variants of the DNA sequences shown in the sequence listing, including those containing silent mutations and those in which mutations result in amino acid sequence changes, are within the scope of the present invention, as are proteins which are allelic variants of SEQ ID No. 2 and Nos. 4, 6, 8, 10, 37, 39, 41 and 43.

The present invention also comprises polynucleotides encoding homologous Type II GnRH-Rs from other species, preferably other mammalian species. Murine, porcine, ovine, bovine, canine, feline, equine and primate Type II GnRH-Rs are of particular interest. The sequence information provided in SEQ ID Nos. 1, 3, 5, 7, 9, 36, 38, 40 and 42 together with the techniques described herein and the standard conventional cloning techniques known in the art are sufficient to obtain such homologous polynucleotides. For example, there is a substantial body of knowledge concerning the techniques required for the art of genetic engineering and reference is made to Maniatis et al, Molecular Cloning, A Laboratory Manual, Cold Spring Harbor Laboratory, Cold Spring Harbor, N.Y. 1982 and “Principles of Genetic Engineering”, Old and Primrose, fifth addition, 1994.

The present invention also includes modified sequences retaining Type II GnRH-R function (i.e. the ability to bind GnRH Type II) and having at least 70% homology (preferably 80% homology, especially preferably 85-90% homology and most preferably over 90% homology) with the nucleotide sequence in question. Functional equivalents of such polynucleotides are also part of this invention. In particular, we include nucleotide substitutions which do not affect the amino acid expressed. The term “functional equivalent” used herein refers to any derivative in which nucleotide(s) and/or amino acid(s) have been added, deleted or replaced without a significantly adverse effect on expression of the gene product or on biological function thereof. Thus, for example, amino acid Glu may be encoded by the codon gag or by the codon gaa and each construct may be varied in this way without affecting the sequence of the expressed peptide. Additionally, most vertebrates have three types of GnRH and it is postulated that three cognate receptors exist. Together with collaborators (Troskie, Kwon and Wangli) we have identified Types I, II and III GnRH receptors in Xenopus and bullfrog. The Type III GnRH-R is more similar to Type II GnRH-R than Type I. We therefore propose the existence of a Type III human GnRH-R with homology to Type II. Thus, the Type III GnRH-R is expected to have substantial homology with the Type II GnRH-R described herein and hence will be covered by the modified versions of the sequences disclosed.

The polynucleotides may be in any form (for example DNA or RNA, double or single stranded) but generally double stranded DNA is the most convenient. Likewise the polynucleotides according to the present invention may be part of a recombinant genetic construct, which itself may include a vector (for example an expression vector) and eukaryotic vectors (as well as prokaryotic vectors) are of interest. Alternatively, the construct may be incorporated into the genome of a transgenic animal. Any vectors or transgenic animals comprising a polynucleotide as described above form a further aspect of the present invention.

Viewed in a yet further aspect the present invention provides a recombinant expression system able to express the Type II GnRH-R described above. DNA constructs (i.e. a standard vector recombinantly combined with a polynucleotide sequence coding for the Type II GnRH-R of interest) and cells transformed with such constructs are also encompassed by the present invention.

The term “expression system” is used herein to refer to a genetic sequence which includes a protein-encoding region and is operably linked to all of the genetic signals necessary to achieve expression of that region. Optionally, the expression system may also include a regulatory element, such as a promoter or enhancer, to increase transcription and/or translation of the protein encoding region or to provide a control over expression. The regulatory element may be located upstream or downstream of the protein encoding region or within the protein encoding region itself.

In addition to the Type II GnRH-R construct described above, the present invention also provides host cells transformed with such constructs and which may express the biologically active modified gene product.

In a further aspect, the present invention provides a stable cell-line capable of expressing Type II GnRH-R, preferably human Type II GnRH-R, as described above. By “stable” we mean that the cell-line retains its ability to express useful quantities of Type II GnRH-R after several (e.g. 10) generations, with any decrease in the level of Type II GnRH-R expression being sufficiently low not to materially affect the utility of the cell-line.

Desirably the host cell transformed with the construct encoding the human Type II GnRH-R is of mammalian origin, but other cell types may also be useful. Examples include prokaryotic cells (such as E. coli), non-mammalian derived eukaryotic cells (such as insect, yeast or plant cells). Suitable host cells include, for example, COS-1 cells, COS-7 cells, COSM6 cells, CHO cells, BHK cells, GH₃ cells, HEK293 cells and 293EBNA cells.

The present invention also provides isolated peptides comprising a functional Type II gonadotropin-releasing hormone receptor (Type II GnRH-R) peptide. Preferably said peptide comprises at least a portion of exon I.

Preferred embodiments of the peptides according to the present invention include peptides having an amino acid sequence corresponding to the sequence of amino acid nos 1 to 170 of SEQ ID No. 2 (preferably comprising substantially the full sequence as set out in SEQ ID No. 2) or corresponding to the sequence of amino acid nos 1 to 168 of SEQ ID No. 6 or 39, amino acid nos 1 to 165 of SEQ ID No. 8 or 41, or amino acid nos 1 to 155 of SEQ ID No. 10 or 43 (preferably also comprising substantially the full sequence of SEQ ID Nos. 4, 6, 8, 10, 37, 39, 41 or 43).

Desirably, the peptide is able to bind to Type II GnRH and, preferably, is a functional receptor therefor.

The present invention also provides antibodies specific to Type II GnRH-R peptides, preferably antibodies which are specific to the extracellular domains of Type II GnRH-R, for example EC3 in exon 3 thereof. The term “antibodies” as used herein includes not only monoclonal and polyclonal antibodies, but also antigen binding fragments thereof, trimeric or tetrameric constructs and recombinant or proteolytic antibody fragments.

Antibodies directed to EC1, EC2 and EC3 (especially EC2) are of particular interest and may be of therapeutic value for cancer treatment. The Type II GnRH-R can be expressed and used to screen agents of potential therapeutic interest.

Thus, the present invention further provides a method of screening an agent for pharmacological activity (i.e. to ascertain its utility in binding to Type II GnRH-R), said method comprising:

a) providing a Type II GnRH-R peptide as described above and exposing said Type II GnRH-R peptide to the agent to be tested; and

b) ascertaining whether said agent interacts with (for example binds specifically to) said Type II GnRH-R peptide.

Labelled (eg. radio-labelled or fluorescent-labelled) antibodies may be used to determine whether the agent and Type II GnRH-R interact together. Optionally a washing step may be included to remove labels not bound to the agent or GnRH-R.

An expression system able to produce the Type II GnRH-R described above may be used in this method; preferably the expression system will be a transformed cell-line, the host cell usually being of mammalian origin. Alternatively the expression system may be a transgenic animal.

Optionally, the receptor expressed may be mutated to produce constitutively active receptors which can be used to screen for antagonists and agonists.

An expression system able to produce the Type II GnRH-R described above may be used to screen agents of potential therapeutic use (such as Type II GnRH agonists or antagonists). Desirably, therefore the expression system will imitate at least some aspects of Type II GnRH-induced signal transduction. Optionally, the expression system may be a stable cell line, the host cell usually being of mammalian origin. Alternatively the expression system may be a transgenic animal.

In another embodiment of the invention, the Type II GnRH-R itself or extracellular domain thereof (e.g. the EC2 or EC3 loop) could be administered in vivo. The free GnRH-R or extracellular domains (which could be synthetic peptides) could competitively bind to GnRH and inhibit its reaction with the native receptor in vivo.

Alternatively, the Type II GnRH-R or an extracellular domain thereof may be used as a means of contraception. For example, a patient may be immunised by injection with the Type II GnRH-R. This will induce antibody production to the Type II GnRH-R and the antibodies so produced will also interact with native Type II GnRH-R affecting reproductive ability. Alternatively, the exogenous Type II GnRhH-R may bind competitively for endogenous GnRH.

### BRIEF DESCRIPTION OF THE DRAWINGS

The present invention will now be further described with reference to the following, non-limiting, examples and Figures in which:

FIG. 1 is a schematic representation of the human gene structure of Type I GnRH-R and of Type II GnRH-R. The positions of the extracellular (EC) and intracellular (IC) loop domains, transmembrane (dark blocks) and carboxy terminal tail (C) are indicated. The approximate positions of the “short splice” (SS) for intron 1′ and selenocysteine (SeCys) in the human receptor is shown.

FIG. 2 shows the immunolocalisation of Type II GnRH receptor and Luteinizing Hormone (LH) to sheep pituitary:

a) immunolocalisation of Type II GnRH-R using antibody against EC3;

b) immunolocalisation of LH to pituitary gonadotropes;

c) co-localisation using antibodies a) and b) and enzymatic detection;

d) co-localisation of Type II GnRH-R (black) and LH (grey) by confocal microscopy; some cells contain LH only but most expressing Type II GnRH-R are LH positive.

FIG. 3 is a comparison of the amino acid sequences of the first human Type II GnRH-R according to splicing alternative 1, and marmoset Type II GnRH-R.

The numbering used is that of the human sequence. Asterisks (*) indicate identity, and vertical slashes (|) conserved substitution. Regions predicted to be helical from homology modelling with the rhodopsin crystal structure are indicated. Exon boundaries, the position of the short splice (intron 1′) and the apparent stop codon in the human sequence are also shown.

The amino acid sequence of the second human Type II GnRH-R sequence according to the first splice alternative (SEQ ID No. 39) may differ from that in FIG. 3 (i.e. SEQ ID No. 6) by 3 amino acids; C71→S, L210→F and N267→I.

FIG. 4 shows the heterologous competitor binding of ¹²⁵I-[His⁵, D-Tyr⁶]GnRH mediated by the Marmoset Type II GnRH-R expressed in COS-7 cells in the presence of increasing doses of the peptides (IC₅₀ values indicated in nM): mGnRH (mammalian GnRH, ▪, 51.7±1.9 nM; GnRH II (GnRH Type II, ▴, 1.3±0.2 nM); and sGnRH (salmon GnRH, ▾, 9.8±1.4 nM). Error bars represent s.e.m. (n=2).

FIG. 5 shows the total inositol phosphate production mediated by the Marmoset Type II GnRH-R expressed in COS-7 cells in response to concentration of various peptides (EC₅₀ values indicated in nM) mGnRH (mammalian GnRH, ▪, 33.2±6.3 nM); GnRH II (GnRH Type II, ▴, 0.46±0.12 nM); sGnRH (salmon GnRH, ▾, 8.7±0.5 nM); [D-Arg⁶] GnRH II ([D-Arg⁶]GnRH Type II, ♦, 1.1±0.23 nM); and Ant 135-18 (Antagonist 135-18, ●, 223±7 nM). Error bars represent s.e.m. (n=2).

FIG. 6 shows receptor binding (a) and inositol phosphate production (b) of mammalian GnRH I (◯) and GnRH II (●) in COS-7 cells transfected with marmoset Type II receptor (left panel) and human Type I receptor (right panel). Stimulation of inositol phosphate by Type I receptor Antagonist 135-18 (□) at the Type II receptor is also shown. Error bars represent s.e.m. of 3-6 separate experiments.

FIG. 7 shows the Luteinizing hormone (LH) and Follicle Stimulating Hormone (FSH) response to GnRH I and II in sheep. FIG. 8 shows activation of ERK2 and p38α MAP kinases by Type I (open bars) and Type II (closed bars) GnRH receptors in COS-7 cells. Stimulation of Type I (a) and II (b) GnRH receptors with mammalian GnRH I, GnRH II and Antagonist 135-18 of immunoprecipitated myc-ERK2. Inset panels depict anti-phospho-ERK2 immunoblotting of anti-myc COS-7 cell immunoprecipitates. FIG. 8c, Left panel,shows selective and time-dependent activation of p38α by GnRH II stimulation (100 nM) of Type II GnRH receptor. Stimulation of Type I GnRH receptor (100 nM) failed to activate p38α MAP kinase. The right panel demonstrates that both Type I and Type II GnRH receptor stimulation induces a time-dependent activation of ERK but that the GnRH II stimulation of the Type II receptor is more prolonged. Data represent mean±s.e.m., n≧3.

FIG. 9 shows expression of Type II GnRH receptor in marmoset and human tissues. (a) RT-PCR was carried out with specific primers on cDNA prepared from marmoset RNA isolated from various tissues. PCR products were fractionated by size on agarose gels. Type II GnRH receptor levels were normalised to actin RNA and represented as the log of the RNA expression relative to pituitary. Hatched bars indicate marmoset brain tissues, solid bars indicate marmoset reproductive tissues while open bars indicate other marmoset tissues. (b, c) Expression of the Type II GnRH receptor in human tissues was examined in Northern blots of mRNA (Clontech) by hybridisation with ³²p labelled human exon 1; (b) mRNA from human cerebellum (1), cerebral cortex (2), medulla (3), spinal cord (4), occipital pole (5), frontal lobe (6), temporal lobe (7) and putamen (8); (c) mRNA from heart (1), whole brain (2), placenta (3), lung (4), liver (5), skeletal muscle (6), kidney (7) and pancreas (8). Another blot showed moderate expression in the amygdala and low expression in caudate nucleus, corpus callosum, hippocampus, substantia nigra, subthalamic nucleus and thalamus (data not shown).

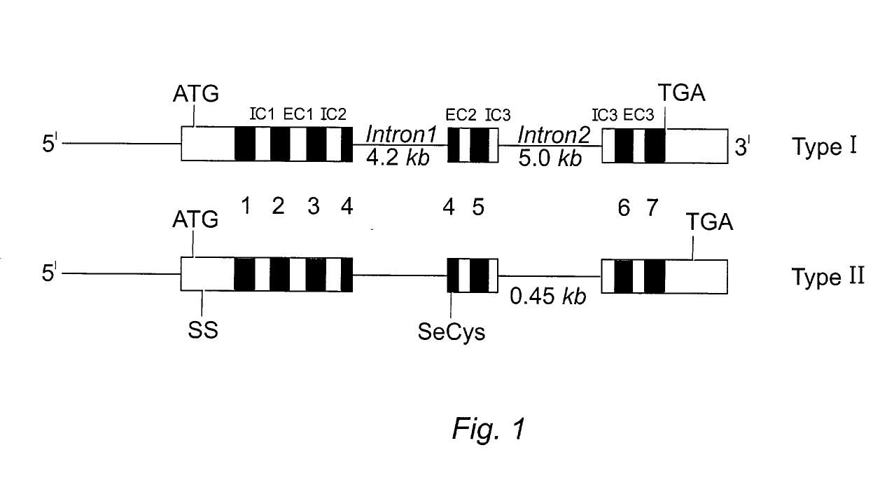

### EXAMPLES

Following numerous failed attempts to locate exon I from the sequence reported by Millar et al. (Journal of Endocrinology (1999), Vol 162, pages 117-126) subsequent work demonstrated that the antisense sequence of Millar et al. was the 3′ untranslated end of an RNP which localised to chromosome 14.

It was recognised that as long as exon 2 or exon 3 sequence information was used in PCR we would always detect the 3′ untranslated end of the RNP since this is highly expressed in all tissues. In addition, the putative intron 2 of Type II receptor would always be retained. Since this transcript ends with a polyadenylation in the region of the equivalent of intron 1 of the putative Type II receptor (read in the opposite direction to RNP), we would never obtain information on exon 1 of the Type II receptor.

We concluded that three new elements were necessary to find the putative Type II GnRH receptor.

a) Search the data bases for a nucleotide sequence encoding part of exon 1.

b) Generate an antibody against a highly specific domain which is responsible for ligand selectivity (see Sealfon et al., Endocr. Review 18:180-205 and Troskie et al., General and Comparative Endocrinology 112:296-302 (1998)) in extracellular loop 3 of the human Type II vis-à-vis Type I receptor to determine in which tissue this rare receptor is expressed.

c) Undertake chromosomal localisation to determine if the coding sequence exists in a pseudogene locale (with RNP) and in another “real” gene location elsewhere.

### Materials and Methods

Cloning of the marmoset Type II GnRH receptor. RNA was isolated from marmoset pituitary and brain stem using RNAsol B (Biogenesis). 2 μg of RNA was incubated with 1 mM dNTPs, 2 μM random hexa-polynucleotides (Promega), gene specific primers or anchored oligo-dT primers at 80° C. for 10 min. 1×RT buffer (Sigma), 1 U/μl RNAsin (Promega) and 0.5 U/μl AMV reverse transcriptase (Sigma) were added in a total volume of 20 μl and incubated at 55° C. for 2 h, then 65° C. for 10 min. Primers designed to the Type II marmoset GnRH receptor exon sequences. Primers were chosen to span putative introns, to enable detection of processed RNA in the presence of possible genomic DNA contamination and the RNP antisense transcript. 50 ng of purified (Qiagen) cDNA produced with random hexa-polynucleotides were subjected to PCR using human sequences previously described (17) and human genomic sequence encoding exon 1 (Zymogenetics AL 160282, BG 636291, AA 954764). Round 1 PCR: 5 cycles at 65° C., 23 cycles at 63° C. using primers S1 and A1 (S1, GATGCCACCTGGAATATCACTG (SEQ ID No. 17); A1, AGGCAGCAGAAGG (SEQ ID No. 18). Round 2 PCR: 5 cycles at 63° C., 25 cycles at 61° C. using 1 μl of products from Round 1 as template and primers S2 and A2 (S2, CAGCCTGGGGACTTAGTTTCCTG (SEQ ID No. 19); A2,GGTTATAGGTGGTCTCTTGC (SEQ ID No. 20). Products were size-purified (Qiagen), cloned into pGEM-T (Promega) and sequenced. Sense and antisense oligonucleotides were designed from the novel marmoset sequences and used in 3′ and 5′ RACE. For the remaining PCRs a three-step protocol was used where the annealing temperature of the first 5 cycles was 2° C. higher than the lower Tₘ of the two primers. In the second step the annealing temperature of 5 cycles was the same as the lower Tₘ of the two primers. The third step was 20 cycles with annealing temperatures 2° C. below the lower Tₘ of the two primers. For the 5′ RACE a poly-A sequence was added to 50 ng marmoset pituitary cDNA produced with gene specific primers. Products were purified (Qiagen) and subjected to PCR. 2 μl of products of a first round PCR, using primers S3 and A3, were used in a second round of PCR using primers S4 and A4 (S3, GACCACGCGTATCGATGTCGACTTTTTTTTTTTTTTTTV (SEQ ID No. 21) (anchored RACE primer from Boehringer Mannheim); A3, GAAGGGACTGGACCAGCTCG (SEQ ID No. 22); S4, GACCACGCGTATCGATGTCGAC (SEQ ID No. 23); A4, CAAGGCAAGCAGGAAACTAAG (SEQ ID No. 24)). For the 3′ RACE 50 nq marmoset pituitary cDNA was produced with anchored oligo-dT primers was subjected to PCR using primers S5 and S3 (S5, ACCTCTTCACCTTCTGCTGCCT (SEQ ID No. 25)). 2 μl of products from this PCR and primers S6 and S4 were used in a secondary PCR (S6, CCTCCTCAATGCTCCTTTGGATC (SEQ ID No. 26). Products were size purified (Qiagen), cloned into pGEM-T (Promega) and sequenced. Full length marmoset Type II GnRH receptor was produced by PCR using oligos of the 5′ UTR (S7, GAATTCGCTTCATACTCACACTTCATC (SEQ ID No. 27); S8, CGGAATTCTCACACTTCATCCTCCTATCTC (SEQ ID No. 28)) and the 3′ sequence including the stop codon (A5, GCTCTAGAGATCAGATTGATGTTATAGGAATG (SEQ ID No. 29)). 50 ng of marmoset brain stem cDNA produced with random hexa-polynucleotides was subjected to PCR using primers S7 and A5. 2 μl of products and primers S8 and A5 were used in a secondary round of PCR. Products of this PCR were purified (Qiagen), cloned into pcDNA3.1+ (Invitrogen) and sequenced. The resultant plasmid was used in expression studies.

Immunocytochemistry. Tissues from human, mouse, sheep, rhesus and cynomologus monkeys were obtained. An antiserum to the human Type II GnRH receptor was produced by immunisation of rabbits with a synthetic peptide corresponding to EC3 (YSPTMLTEVPPC (SEQ ID No. 30)) conjugated to keyhole limpet haemocyanin via the Cys residue. This peptide, a synthetic peptide to EC3 of the human Type I receptor (DPEMLNRLSDPC (SEQ ID No. 31)) and haemocyanin were used for immunoneutralisation specificity studies. For detection of mammalian GnRH I specific antiserum GF6 was used (18).

Tissue sections (15 μm) were subjected to the peroxidase/diaminobenzidine visualisation technique as previously described (18, 19). Fluorescent labelling was accomplished using the same procedure up to the step prior to ABC reaction, when the fluorescein label (Rhodamine 600, avidin D or FITC) was applied to the slides and incubated at room temperature in the dark for 2-4 hours. For double labelling, slides were incubated sequentially with avidin D and biotin blocking solutions for 15 min each, then re-incubated with the next primary antibody, followed by the other fluorescent labelling (Rhodamine or FITC). Controls, including omission of primary antibodies and order of exposure, were consistently negative. Immunofluorescence was viewed at two wave lengths by confocal microscopy.

Cell culture and transfection. COS-7 cells were cultured as previously described (20, 21). Transient transfections of COS-7 cells with human Type I GnRH receptor or marmoset Type II GnRH receptor, along with myc-tagged ERK2, JNK and p38α constructs were performed using Superfect (Qiagen) according to the manufacturer's protocol. All assays described were performed 48 h post transfection.

Receptor binding and inositol phosphate production. Receptor binding utilising ¹²⁵I-[His⁵-D-Tyr⁶]GnRH I and inositol phosphate production by GnRH ligands were studied as previously described (20-22).

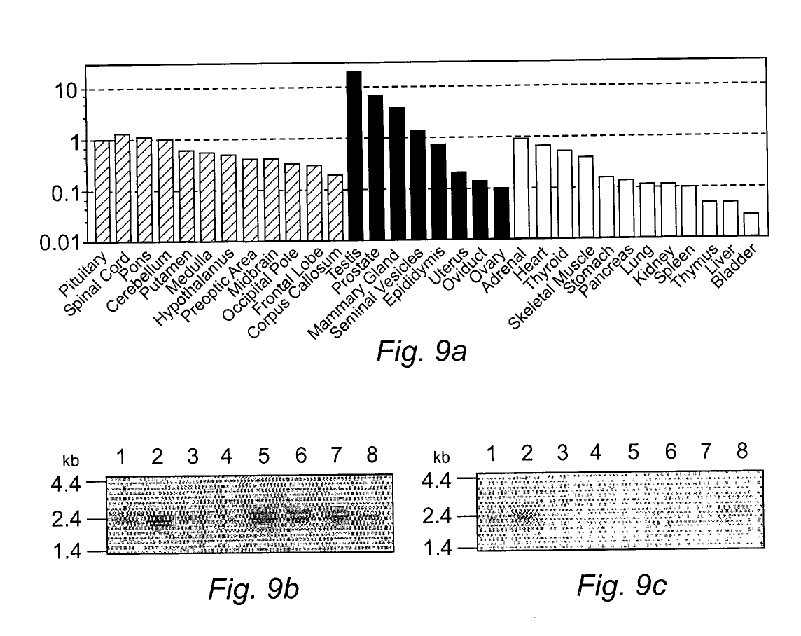

Phospho-MAP kinase assay. Serum starved (12-16 h) COS-7 cells, transfected with human Type I or marmoset Type II GnRH receptors were treated with either mammalian GnRH I, GnRH II or antagonist 135-18 for the time and dose specified for FIG. 9 at 37° C. After ligand stimulation, COS-7 cells were prepared for immunoprecipitation (23). Co-transfected myc-tagged MAP kinase constructs were immunoprecipitated from cell lysates by overnight incubation with myc-agarose slurry (Santa Cruz), washed, an equal volume of 2×Laemmli sample buffer added, resolved by SDS-PAGE and electrotransferred to PVDF membrane (NEN Life Sciences). Activated MAP kinase was detected using anti-phospho-ERK/JNK/p38a-kinase specific antisera (New England Biolabs), visualised by enzyme-linked chemifluorescence (Amersham-Pharmacia) and quantified using a phosphorimager. The degree of phosphorylated MAP kinase was normalised to the amount of unphosphorylated MAP kinase detected with specific antisera.

Expression of Type II receptor mRNA in marmoset and human tissues. Total RNA was extracted from various marmoset tissues using TRI reagent (Sigma), and cDNA was produced using oligo dT primers (Ambion). PCR was performed on the cDNA using marmoset Type II GnRH receptor cDNA specific primers spanning exons 1-3 (sense: CTTCGGCTGGAGGGAACCTG, antisense: GGTGCCCTCTTCGGCAGC), and actin specific primers. PCR products were run on an agarose gel and blotted onto HybondN⁺ nylon membrane (Amersham). The Southern blot was probed with random primed marmoset Type II GnRH receptor cDNA or actin cDNA. Southern blots were quantified using a phosphorimager and marmoset Type II GnRH receptor expression was normalised to the expression of actin.

Expression in human tissues was examined by Northern blots of mRNA with random primed ³²p labelled human Type II GnRH receptor exon 1. The human Type II GnRH receptor genomic sequence (P1 clone) was obtained by Genome Systems (St Louis, Md.) using PCR screen of P1 clones with oligonucleotides to human sequences (17). Oligonucleotides (antisense: CTGTCCTGCCCGGTCCTGAG; sense: TGCCCACCTTCTCGGCAGCA) to this sequence were used with the P1 clone to produce a 460 bp amplicon. Labelling was done with ³²p dCTP (6000 Ci/mMole) using supplier's specified conditions (Stratagene). Hybridisation was performed using 2×10⁷ cpm at 65° C. in 5×SSC/0.005% SDS/5×Denhardt's/2 mg/ml salmon sperm DNA, washing with 0.1×SSC/0.5% SDS at 55° C. and the blots exposed to X-ray film for 6 days.

Stimulation of LH and FSH in sheep. The relative potency of mammalian GnRH I and GnRH II at inducing FSH and LH secretion in vivo was tested using our Soay ram sheep model (24). This was repeated during both the sexually active (short days, SD) and inactive phases (long days, LD) of the photoperiod-induced reproductive cycle (24). The same animals (n=8) were used on the two occasions. The GnRHs were administered at doses of 250 ng/ram and 10 μg/ram in a cross-over design with a week between treatments to permit full recovery. The peptides were dissolved in 1 ml of 0.9% saline and given as an intravenous bolus. Blood samples were collected every 10 min, from 20 min before, until 2 h after the treatments and assayed by specific radioimmunoassays (24). The FSH and LH responses to the GnRH agonists were calculated as delta responses (2 h mean hormone concentration post treatment minus 20 min mean pre-treatment hormone concentration). These values were used to calculate the FSH:LH response ratio for GnRH II and GnRH I, and thus the overall ratio for the GnRH II stimulation compared with the mammalian GnRH I stimulation. This was assessed for each animal and then for the group (mean±SEM, n=8), under both LD and SD.

### Results and Discussion

Cloning and Primary Structure of the Marmoset Type II GnRH Receptor.

Amplification of EC3 from genomic DNA from a range of vertebrate species revealed two distinct sequences of receptors representing the known Type I receptors and novel Type II receptors in an amphibian and reptile (25). Searches of human EST databases revealed homologous sequences to the reptile EC3 (17). From EST contigs we constructed a partial receptor sequence encoding the putative exons 2 and 3 corresponding to these exons of the Type I receptor (17). All ESTs were in the antisense orientation and it transpired that these were in the 3′ untranslated region (UTR) of a novel human ribonucleoprotein (RBM8) which was highly expressed in all tissues examined (26). The equivalent of exon 1 was absent from the RMB8 3′ UTR. It was therefore evident that the identification of sequences homologous to exon 1 was essential to discover the Type II receptors. Searches of human databases, using as a query exon 1 of the human Type I receptor, revealed several ESTs and genomic sequences BG 036291, AA 954764; AL

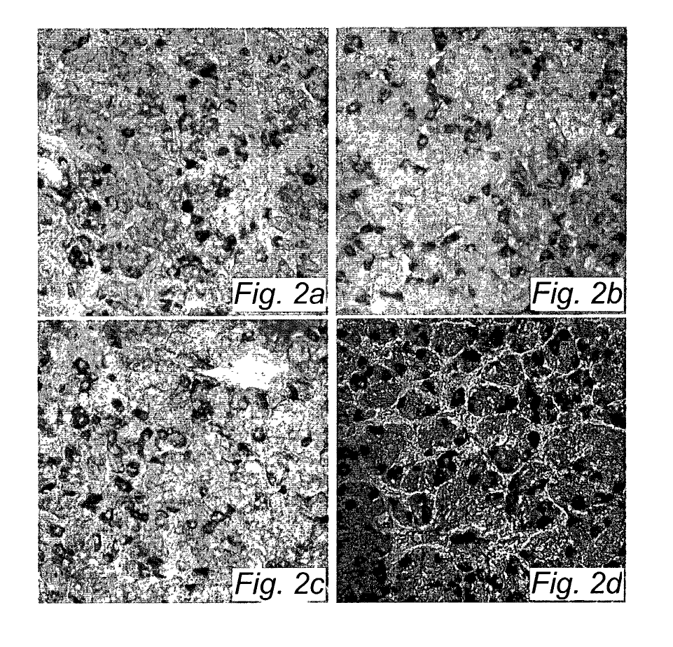

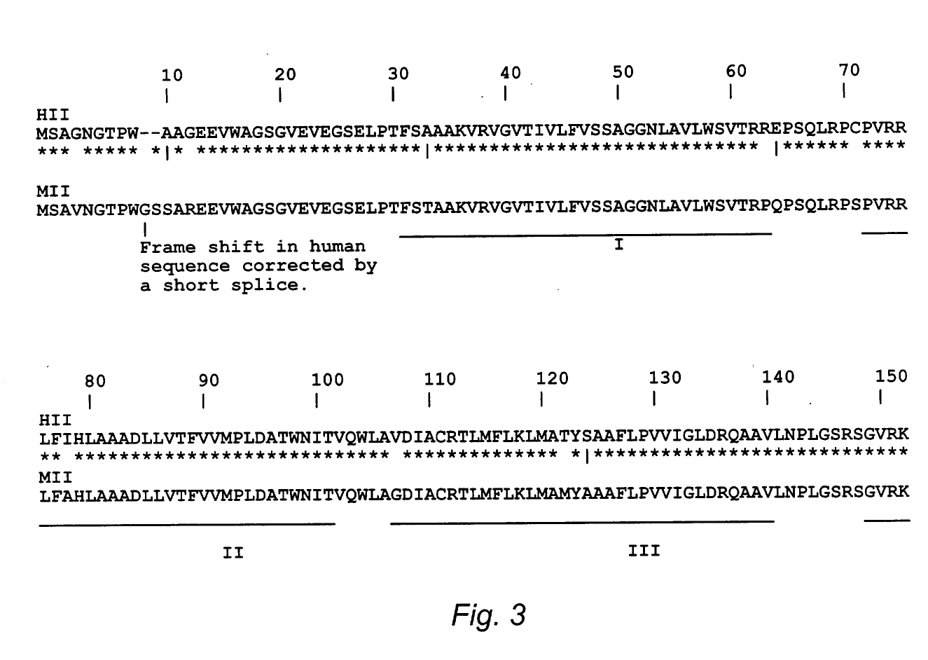

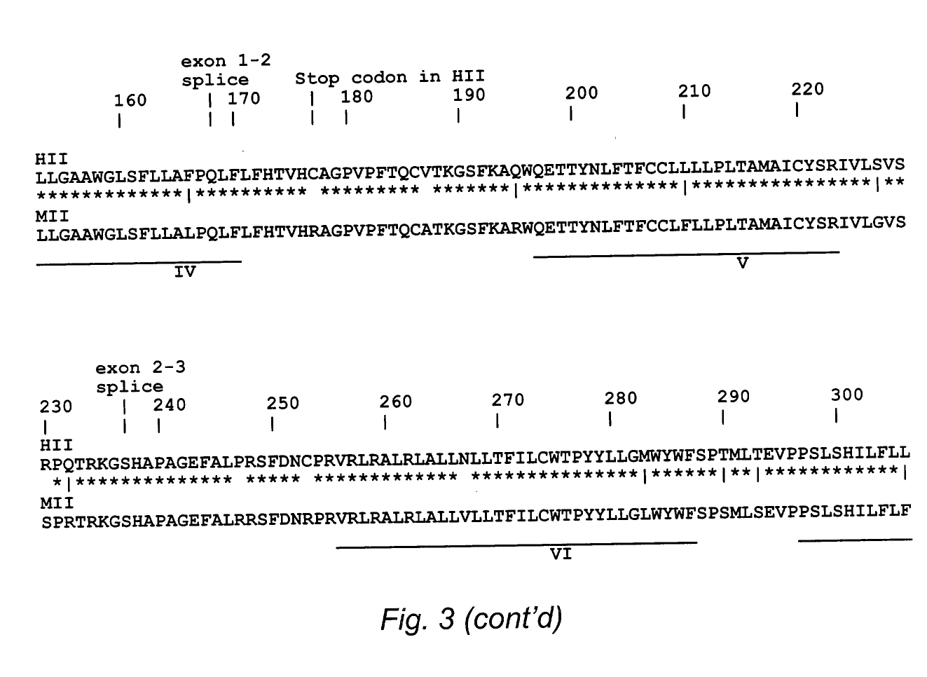

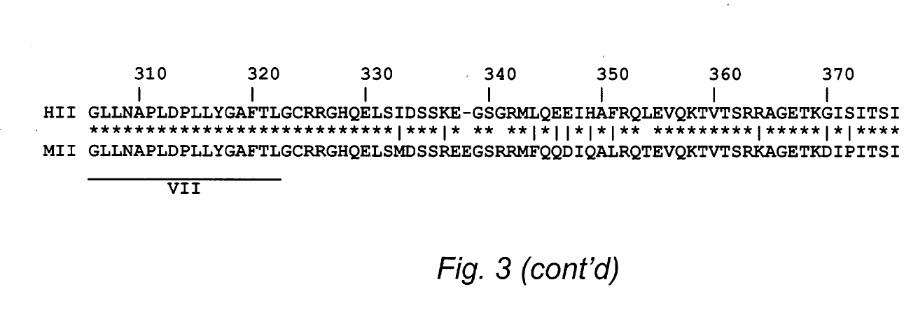

Since the receptor was likely to be a rare transcript expressed in discrete tissues, we generated antisera to the EC3 domain of the human Type II receptor and found by immunocytochemistry strong reactivity in pituitary and brain of the human, rhesus monkey, sheep and mouse (FIG. 2). We then used oligonucleotides to the human exon 1 and RBM8 3′ UTR sequences to amplify cDNA from marmoset pituitary and brain by PCR and 5′ RACE procedures. The full length cDNA encodes a 380 amino acid protein with characteristic G protein-coupled receptor (GPCR) structure SEQ ID No. 1. Although it is more homologous with GnRH receptors than other GPCRs, it has only 41% sequence identity with the human Type I receptor suggesting an early evolutionary gene duplication. It also possesses a carboxy terminal tail which is important for rapid desensitization and is uniquely absent from mammalian Type I receptors (27-30). The receptor also does not have the unusual Asn/Asp microdomain of transmembrane helices 2 and 7 of the mammalian Type I receptors which plays a role in receptor activation (21). Instead it has the Asp/Asp motif as in non-mammalian Type I GnRH receptors recently cloned (7, 9). The Drosophila GnRH receptor homologue has the usual Asp/Asn motif characteristic of most GPCRs (31) indicating that there was an initial mutation to Asp/Asp in the ancient vertebrate GnRH receptor followed by mutation to Asn/Asp in the mammalian Type I receptors. The activation role of this microdomain (21) may therefore be further elucidated by experimentation with the Type II receptor. The LSD/EP sequence of EC3 which is important for ligand selectivity of mammalian Type I receptors (9, 20) is replaced by VPPS which is also present in reptile (VPPS) and amphibian (VPPV) Type II GnRH receptors (25). This difference in sequence is likely, therefore, to be a determinant of Type II receptor selectivity for GnRH II as all other binding sites (9) are conserved.

Pharmacological Characterization of the Marmoset Type II GnRH Receptor.

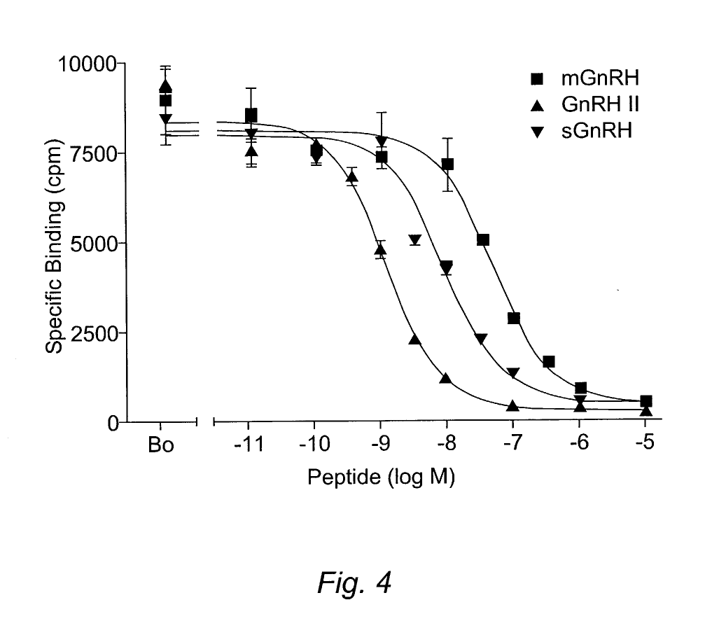

FIG. 4 shows the heterologous competition binding of ¹²⁵I-[His⁵, D-Tyr⁶]GnRH mediated by the Marmoset Type II GnRH receptor expressed in the transfected COS-7 cells and shows that the Type II GnRH receptor demonstrates high selectivity for GnRH Type II.

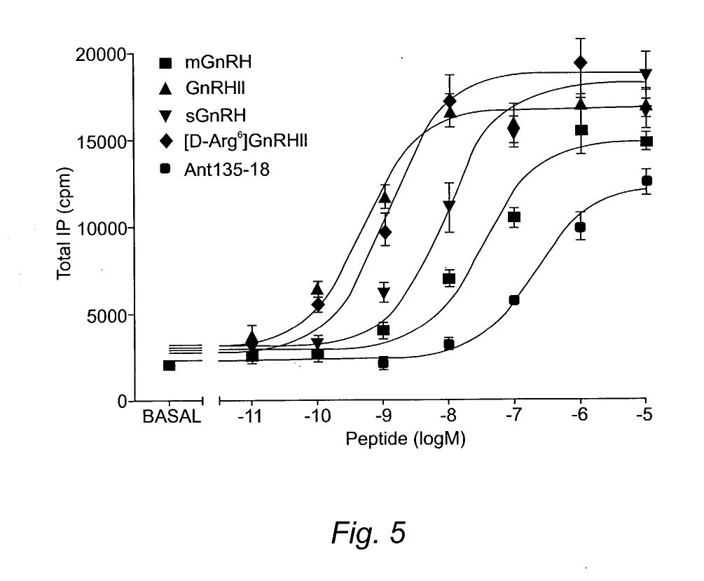

The affinity and specificity of the Type II GnRH receptor for the natural GnRHs and two GnRH analogues is shown in FIG. 5. Further characterisation of GnRH agonists and antagonists can be determined with the routine binding assays. The identification of the signalling pathway of the receptor was examined by testing the ability of the ligand-induced receptor to activate second messenger generating systems.

Using the above described expression system, we have determined that the Type II GnRH-R activates the production of Inositol phosphate (see FIG. 5) but does not stimulate cyclic AMP production.

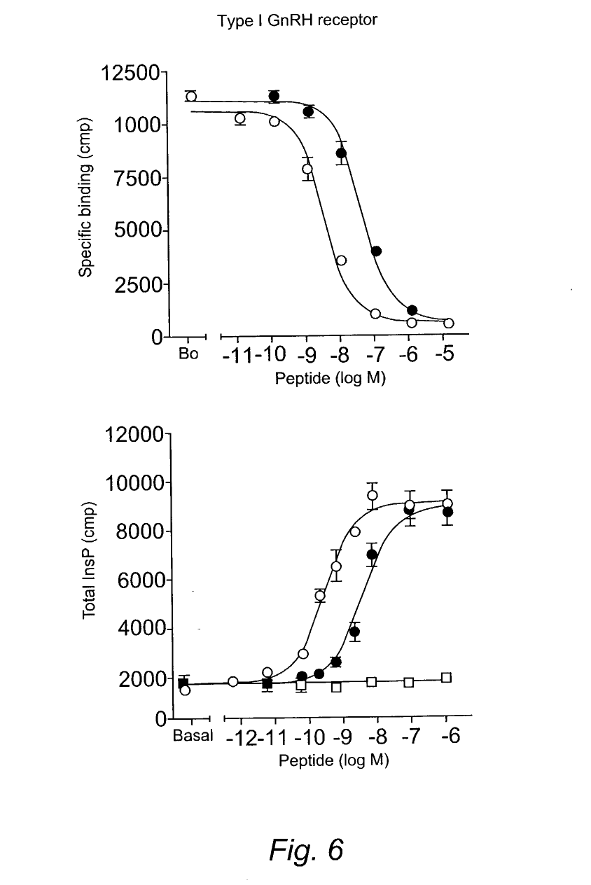

Thus, expression of the Type II receptor in COS-7 cells revealed that it is highly selective for GnRH II in receptor binding assays (FIG. 4) and in the stimulation of inositol phosphate intracellular messenger production (40-fold and 90-fold greater activity relative to mammalian GnRH I) (FIG. 5) (Table 1). This contrasts with the Type I receptor in which GnRH II has only 10% and 9% activities of mammalian GnRH I in these assays (FIG. 6). Overall GnRH II has an affinity 24-fold greater for the Type II receptor than for the Type I receptor. The Type II receptor was also more selective for salmon GnRH and [D-Arg⁶]GnRH II (Table 1).

TABLE 1
Comparative ligand binding and inositol
phosphate production properties of marmoset type II &
human Type 1 GnRH receptors.

| | Ligand binding | | InsP production | |
|---|---|---|---|---|
| | (IC₅₀) | | (EC₅₀) | |
| | Marmoset | Human | Marmoset | Human |
| Peptides | Type II | Type I | Type II | Type I |
| GnRH II | 1.07 ± 0.04 | 26.1 ± 4 | 0.45 ± 0.05 | 7.41 ± 1.55 |
| GnRH I | 42.6 ± 3.19 | 2.81 ± 0.17 | 40.5 ± 4.43 | 0.63 ± 0.08 |
| sGnRH | 9.48 ± 2.17 | 244 ± 23.6 | 5.99 ± 0.91 | 9.62 ± 3.5 |
| [D-Arg⁶] | 3.34 ± 0.06 | 11.9 ± 0.35 | 2.39 ± 0.64 | 3.8 ± 0.71 |
| GnRH II | | | | |
| Antagonist | 1650 ± 478 | 10.6 ± 1.4 | 276 ± 45.5 | Full |
| 135-18 | | | | antagonist |

Data are expressed in nanomolar and represent the s.e.m. (n = 3-6). Mammalian GnRH I (GnRH I) salmon GnRH (sGnRH: [Trp⁷,Leu⁸]GnRH I), GnRH II ([His⁵,Trp⁷,Tyr⁸]GnRH I)

Moreover, a Type I receptor GnRH antagonist behaved as an agonist at the Type II receptor (FIG. 5). It has recently been demonstrated that control of gonadotropin biosynthesis and regulation of gonadotrope function regulated by GnRH can be mediated by the activation of mitogen-activated protein kinases (MAP kinases). Therefore we assessed the capacity of both the Type I human GnRH receptor and the marmoset Type II GnRH receptor to activate the three major MAP kinase prototypes, ERK, JNK (a particular type of MAP kinases available through Eisuke Nishida, Kyoto University) and p38α in COS-7 cells. At the Type I receptor, mammalian GnRH I was considerably more potent than GnRH II in activating ERK2 (FIG. 8a). In contrast, at the Type II receptor the ligand specificity was the inverse and antagonist 135-18 had significant agonist activity compared with low activity at the Type I receptor (FIG. 8 panels a and b). Agonist-induced activation of JNK was not detected with stimulation of either the Type I or Type II receptor (data not shown). However, activation of p38α was detected upon stimulation of the Type II receptor but not with stimulation of the Type I receptor (FIG. 8c). The time course of p38α activation was also considerably more protracted than that for Type I/II receptor activation of ERK2. In addition we noted that ERK2 stimulation via the Type I GnRH receptor is more transient than that mediated by Type II GnRH receptor stimulation (FIG. 8c). There are therefore distinct differences in signalling by the two receptors.

Tissue distribution and expression of the marmoset Type II GnRH receptor. To gain insight into the potential functions of the Type II receptor, its expression in human and marmoset tissues was examined. PCR amplification of cDNA from marmoset brain tissues revealed that it is expressed in the pituitary, spinal cord, pons, cerebellum, putamen, medulla, hypothalamus, preoptic area, midbrain, occipital pole, frontal lobe and corpus callosum (FIG. 9a). Expression was high in reproductive tissues such as testis, prostate, mammary glands, seminal vesicles and epididymis. Substantial expression was detected in adrenal, thyroid, heart and skeletal muscle but little or no expression was found in other tissues such as liver, ovary and bladder (FIG. 9a). Northern blots yielded a similar expression pattern (data not shown). Northern blots on human tissues probed with exon 1 showed highest expression in the cerebral cortex and occipital pole, moderate expression in the frontal lobe, temporal lobe and putamen, and low expression in the cerebellum, medulla and spinal cord (FIG. 9b). There was substantial expression in the amygdala and low expression in the caudate nucleus, corpus callosum, hippocampus, substantia nigra, subthalamic nucleus and thalamus (data not shown). There was also significant expression in the heart and pancreas but little or no expression in placenta, lung, liver, skeletal muscle and kidney (FIG. 9c).

Type II GnRH receptor function in the pituitary. A specific antiserum to EC3 of the human Type II receptor was used to conduct immunocytochemistry and demonstrated specific expression of the receptor in human anterior pituitary (FIG. 2). In view of the possibility that the novel Type II receptor may regulate pituitary function, we determined if it was expressed in the pituitary of other mammals. Staining was also found in about 10% of cells, (the relative occurrence of gonadotropes), in the anterior pituitary of the mouse and sheep (FIG. 2). In the sheep anterior pituitary double staining with Type II receptor and LH antisera revealed that the Type II receptor immunoreactivity is co-localised in 69% of LH positive cells (FIG. 2). Only 12% of Type II receptor positive cells were negative for LH. Since mammalian GnRH I binding sites also co-localise with LH in up to 90% of gonadotropes in the rat pituitary at proestrus (32), it is likely that the majority of gonadotropes express both Type II and Type I receptors and suggests that these receptors may co-ordinately regulate LH and FSH biosynthesis and secretion. The presence of Type II receptors in the majority of gonadotropes is, at first consideration, unexpected as there is a substantial literature suggesting that a single GnRH (mammalian GnRH I) is sufficient to regulate the secretion of gonadotropins, and that the differential secretion of LH and FSH during the mammalian ovarian cycle may be adequately accounted for by modulatory effects of gonadal steroids (androgen, estrogen and progesterone) and peptides (activin, inhibin and follistatin) (14). However, a substantial number of physiological studies invoke the existence of an FSH-releasing peptide to account for the differential secretion of gonadotropins (13-16).

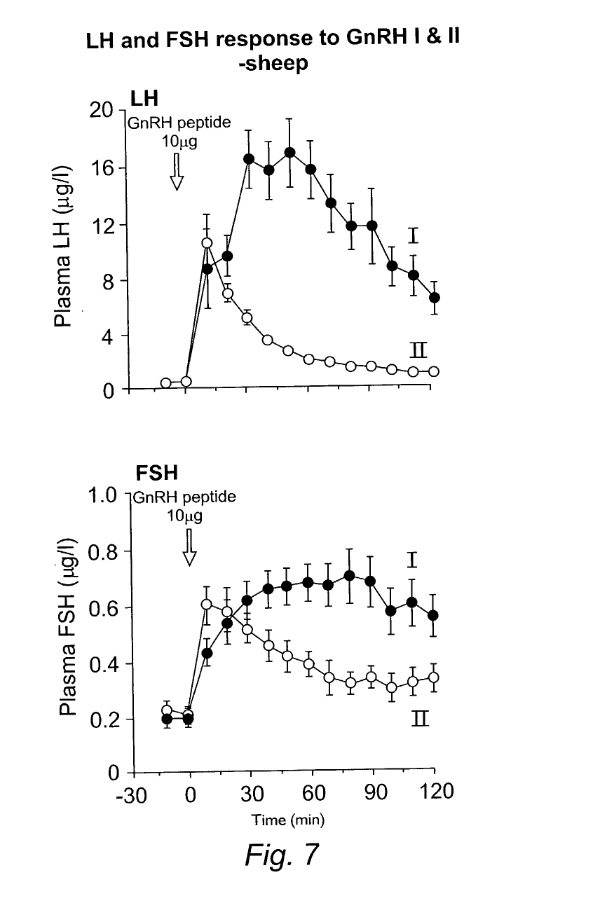

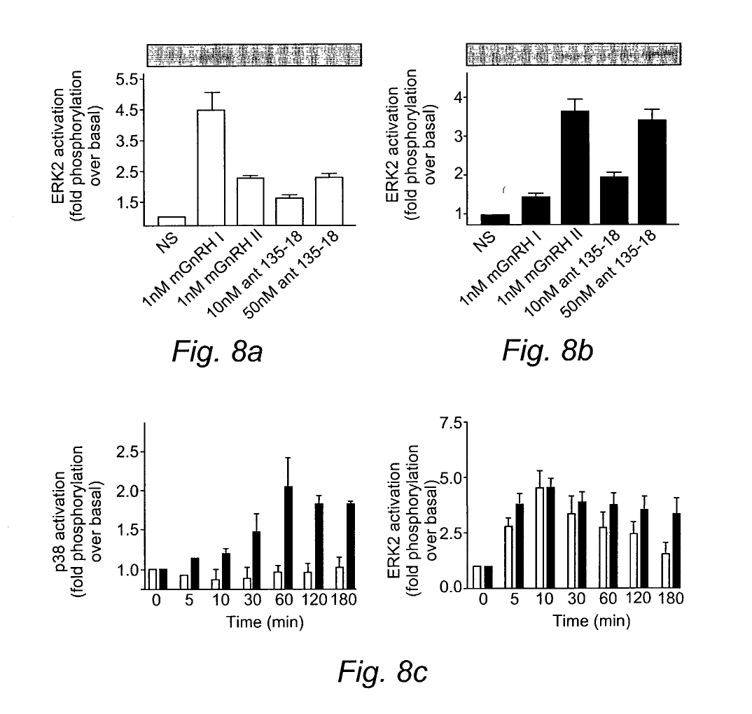

In the early studies on the GnRH II (previously called chicken GnRH II), it was found to have preferential FSH-releasing activity when compared with chicken GnRH I (chicken Type I GnRH) (33). Moreover, GnRH II has been localised to the hypothalamic area in species of non-mammalian vertebrates (see Refs. (7, 8) for review) and the supraoptic, paraventricular, arcuate and pituitary stalk regions of monkeys where it is thought to play a role in gonadotropin secretion (19, 34). We therefore conducted studies using a well-established sheep model to determine the relative effects of mammalian GnRH I and GnRH II on LH and FSH secretion. The responses to a 250 ng bolus of GnRHs was too low for comparison of relative LH and FSH secretion. At the 10 μg dose all the rams showed a robust response and every individual exhibited a higher ratio of FSH to LH secretion when treated with GnRH II compared with mammalian GnRH I (FIG. 7). The mean ratio of FSH to LH induced by GnRH II was 2.14±0.29 and 2.02±0.34 (mean±SD) times higher than that induced by mammalian GnRH I for sexually active and sexually quiescent rams respectively. The FSH/LH ratios generated in both sexually active and quiescent rams upon GnRH II treatment were both significantly greater (p=0.03, p=0.002 respectively, paired two-tailed t-test) than with mammalian GnRH I treatment.

When it is considered that GnRH II has an affinity and potency of ≦20% of mammalian GnRH I at the Type I receptor (7, 9, 20), and the in vivo secretion of the two peptides is likely to be finely tuned in both concentration and phasing of pulsatile release, our exogenous bolus administration of the peptides is a relatively crude approach. Thus, the relative differential stimulation of FSH by endogenously secreted GnRH II may be much greater in vivo.

The findings of GnRH II in the hypothalamus, the presence of the Type II receptor immunoreactivity in gonadotropes and differential release of gonadotropins suggest that, contrary to existing dogma, gonadotropins are regulated by two different forms of GnRH acting through two separate cognate gonadotrope receptors. In order to elicit differences in relative LH and FSH secretion mammalian GnRH I and GnRH II would have to have different patterns of duration of release, concentration and pulse frequency of secretion and/or differential intracellular signalling pathways. The differential, temporal or qualitative, downstream signalling between the Type II and Type I receptors shown here may provide the means for preferential FSH secretion.

GnRH II activation of the Type II receptor in bullfrog sympathetic ganglia potently inhibits M-type K⁺ channels (11). A similar action in gonadotropes would partially depolarise them thus facilitating external excitatory inputs to the cell or entry of extracellular Ca²⁺ through L type channels, which occurs on stimulation of Type I receptors by mammalian GnRH I (1-3). These two GnRHs and GnRH receptor systems, along with differences in signalling pathways, provide the means for differential FSH and LH secretion and open the possibility of developing new GnRH II analogues for the treatment of diseases of the reproductive system as well as contraceptives which selectively inhibit FSH and gametogenesis without affecting sex steroid hormone production.

Type II GnRH receptor may have roles in neural development and sexual arousal. In view of its wide distribution in the central and peripheral nervous systems, GnRH II has been proposed to have a neuromodulatory role (7, 8) as evidenced by K⁺ channel inhibition in bullfrog sympathetic ganglia (11). Our demonstration of a GnRH II-selective receptor expression in many brain regions (FIGS. 2, 9) supports this. Type II GnRH receptor antisera immunoreactive cells were widely seen in the extrahypothalamic regions, such as medial septum, bed nucleus of the stria terminalis, medial preoptic area, substantia innominata, basal nuclues of Meynert, claustrum, amygdala and putamen, and in the hypothalamic regions, such as supraoptic nucleus, periventricular area, ventromedial nucleus and dorsomedial nucleus in rhesus monkeys at embryonic days E58, E70 and E78 as well as in the adult cynomologus monkey. In some of these areas (e.g. midbrain and supraoptic nucleus) the GnRH II ligand is also expressed (19, 34). The distribution pattern of Type II GnRH receptor positive cells in extrahypothalamic regions overlapped with that of the early developing mammalian GnRH I cells we have described (18). Later in embryonic development the GnRH I cells were not consistently immunopositive with Type II GnRH receptor antiserum. This suggests a potential role for the receptor in the development of mammalian GnRH I neurones. An intriguing observation was that neurones which express the mammalian GnRH I gene in the preoptic area and periventricular region of the hypothalamus (Ref. 34) were stained with the Type II GnRH receptor antiserum in the rhesus monkey, suggesting that

GnRH II may regulate mammalian GnRH I neurones. Mammalian GnRH I is known to have ultrashort feedback on its own secretion (8, 35) but the co-localisation of Type II receptor on mammalian GnRH I neurones in the hypothalamic regions suggests that some effects on mammalian GnRH I secretion may be mediated via GnRH II.

GnRH has been shown to have direct effects on reproductive behaviour and sexual arousal in rodents independent of its stimulation of sex hormone production (8, 36). Rapid changes in GnRH content of brain areas, cell number and cell size in response to visual, olfactory and other stimulants of sexual behaviour have been observed in species of fish, amphibians, reptiles and mammals (see Refs. (7, 8, 10, 36, 37) for review). Moreover, GnRH II is much more effective than mammalian GnRH I in stimulating courtship and song in ring doves (7) and song sparrows (12), and GnRH II distribution shifts from midbrain cell bodies to terminal regions following the initiation of courtship in newts (37). There is remarkable concurrence of the distribution of the Type II GnRH receptor in the temporal lobe, putamen, amygdala, medial preoptic area, ventromedial nucleus, dorsomedial nucleus and periventricular nucleus of the human or monkey brain with effects of lesions and/or electrical stimulation of these areas on reproductive behaviours such as sexual interest, erection, intromission, thrusting and ejaculation in rats, dogs, cats, monkeys and humans (36-38). GnRH II has also been localised to these regions in the rhesus monkey (19, 34).

The mechanism of action of GnRH II in the nervous system of mammals is unknown but the peptide has been identified in sympathetic ganglia of amphibians where it binds to selective high affinity receptors (22) and potently inhibits M-type K⁺ channels (11). Inhibition of these K⁺ channels by GnRH II facilitates fast excitatory transmission by conventional neurotransmitters, by increasing input resistance of postsynaptic neurones and by partial depolarization (11). This may, therefore, provide a general neuromodulatory mechanism for GnRH II effects in the nervous system, and specifically in reproductive behaviour, by facilitating signalling by neurotransmitters.

Type II GnRH receptor in reproductive tissues. The marmoset Type II GnRH receptor expression and GnRH II ligand expression (7, 8, 11) in non-neural reproduction-related tissues such as the mammary gland, prostate, gonads and adrenal cells may resolve the long-standing enigma of the non-concurrence of the binding pharmacology of receptors in these tissues and in various tumours (e.g. prostate, ovarian and mammary gland (1-3)) with that of the known pituitary Type I receptor which is believed to be the receptor in these tissues. For example, the paradox of similar effects of both GnRH agonists and antagonists (1, 2) on proliferation of these tumour cell lines can be rationalised if the Type II receptor is mediating these effects, as we have shown that certain mammalian GnRH I antagonists (e.g. 135-18) behave as agonists with the Type II receptor (FIG. 8b). Moreover, the antiproliferative effects of GnRH analogues on cell lines of these tumours is consistent with the activation of p38α by the Type II receptor since this MAP kinase is known to be antiproliferative (39).

### References

1. Millar, R. P., King, J. A., Davidson, J. S. Milton, R. C. (1987) S Afr Med J 72, 748-55.

2. Conn, P. M. & Crowley, W. F., Jr. (1991) N Engl J Med 324, 93-103.

3. Conn, P. M. & Crowley, W. F., Jr. (1994) Annu Rev Med 45, 391-405.

4. Nieschlag, E., Behre, H. M. & Weinbauer, G. F. (1992) in Spermatogenesis-Fertilization-Contraception. Molecular, cellular and endocrine events in male reproduction, eds. Nieschlag, E. & Habenicht, U. -F. (Springer-Verlag, Berlin), pp. 447-501.

5. Fraser, H. M. (1993) Br Med Bull 49, 62-72.

6. King, J. A. & Millar, R. P. (1979) Science 206, 67-9.

7. King, J. A. & Millar, R. P. (1997) in GnRH Neurones: Gene to Behavior, eds. Parhar, I. & Y., S. (Brain Shuppan), Tokyo, pp. 51-77.

8. Sherwood, N. M., Lovejoy, D. A. & Coe, I. R. (1993) Endocr Rev 14, 241-54.

9. Sealfon, S. C., Weinstein, H. & Millar, R. P. (1997) Endocr Rev 18, 180-205.

10. White, R. B., Eisen, J. A., Kasten, T. L. & Fernald, R. D. (1998) Proc Natl Acad Sci USA 95, 305-9.

11. Bosma, M. M., Bernheim, L., Leibowitz, M. D., Pfaffinger, P. J. & Hille, B. (1990) in G Proteins and Signal Transduction (The Rockefeller University Press), pp. 43-59.

12. Maney, D. L., Richardson, R. D. & Wingfield, J. C. (1997) Horm Behav 32, 11-8.

13. Lumpkin, M. D., Moltz, J. H., Yu, W. H., Samson, W. K. & McCann, S. M. (1987) Brain Res Bull 18, 175-8.

14. Padmanabhan, V. & McNeilly, A. S. (2001) Reproduction 121, 21-30.

15. Yu, W. H., Karanth, S., Walczewska, A., Sower, S. A. & McCann, S. M. (1997) Proc Natl Acad Sci USA 94, 9499-503.

16. Yu, W. H., Karanth, S., Sower, S. A., Parlow, A. F. & McCann, S. M. (2000) Proc Soc Exp Biol Med 224, 87-92.

17. Millar, R., Conklin, D., Lofton-Day, C., Hutchinson, E., Troskie, B., Illing, N., Sealfon, S. C. & Hapgood, J. (1999) J Endocrinol 162, 117-26.

18. Quanbeck, C., Sherwood, N. M., Millar, R. P. & Terasawa, E. (1997) J Comp Neurol 380, 293-309.

19. Lescheid, D. W., Terasawa, E., Abler, L. A., Urbanski, H. F., Warby, C. M., Millar, R. P. & Sherwood, N. M. (1997) Endocrinology 138, 5618-29.

20. Flanagan, C. A., Becker, II, Davidson, J. S., Wakefield, I. K., Zhou, W., Sealfon, S. C. & Millar, R. P. (1994) J Biol Chem 269, 22636-41.

21. Flanagan, C. A., Zhou, W., Chi, L., Yuen, T., Rodic, V., Robertson, D., Johnson, M., Holland, P., Millar, R. P., Weinstein, H., Mitchell, R. & Sealfon, S. C. (1999) J Biol Chem 274, 28880-6.

22. Troskie, B., King, J. A., Millar, R. P., Peng, Y. Y., Kim, J., Figueras, H. & Illing, N. (1997) Neuroendocrinology 65, 396-402.

23. Maudsley, S., Pierce, K. L., Zamah, A. M., Miller, W. E., Ahn, S., Daaka, Y., Lefkowitz, R. J. & Luttrell, L. M. (2000) J Biol Chem 275, 9572-80.

24. Lincoln, G. A. & Clarke, I. J. (1998) J Neuroendocrinol 10, 461-71.

25. Troskie, B., Illing, N., Rumbak, E., Sun, Y. M., Hapgood, J., Sealfon, S., Conklin, D. & Millar, R. (1998) Gen Comp Endocrinol 112, 296-302.

26. Conklin, D. C., Rixon, M. W., Kuestner, R. E., Maurer, M. F., Whitmore, T. E. & Millar, R. P. (2000) Biochim Biophys Acta 1492, 465-9.

27. Tensen, C., Okuzawa, K., Blomenrohr, M., Rebers, F., Leurs, R., Bogerd, J., Schulz, R. & Goos, H. (1997) Eur J Biochem 243, 134-40.

28. Illing, N., Troskie, B. E., Nahorniak, C. S., Hapgood, J. P., Peter, R. E. & Millar, R. P. (1999) Proc Natl Acad Sci USA 96, 2526-31.

29. Troskie, B. E., Hapgood, J. P., Millar, R. P. & Illing, N. (2000) Endocrinology 141, 1764-71.

30. Sun, Y. M., Flanagan, C. A., Illing, N., Ott, T. R., Sellar, R., Fromme, B. J., Hapgood, J., Sharp, P., Sealfon, S. C. & Millar, R. P. (2000) J Biol Chem.

31. Hauser, F., Sondergaard, L. & Grimmelikhuijzen, C. J. (1998) Biochem Biophys Res Commun 249, 822-8.

32. Childs, G. V., Unabia, G. & Miller, B. T. (1994) Endocrinology 134, 1943-51.

33. Millar, R. P., Milton, R. C., Follett, B. K. & King, J. A. (1986) Endocrinology 119, 224-31.

34. Urbanski, H. F., White, R. B., Fernald, R. D., Kohama, S. G., Garyfallou, V. T. & Densmore, V. S. (1999) Endocrinology 140, 1945-8.

35. Leranth, C., Segura, L. M., Palkovits, M., MacLusky, N. J., Shanabrough, M. & Naftolin, F. (1985) Brain Res 345, 332-6.

36. Moss, R. L. (1979) Annu Rev Physiol 41, 617-31.

37. Muske, L. E., King, J. A., O'Connell, B. G., Moore, F. L. & Millar, R. P. (1995) Soc. Neurosci. Abstr. 21, 100.

38. Slimp, J. C., Hart, B. L. & Goy, R. W. (1978) Brain Res 142, 105-22.

39. Smalley, K. & Eisen, T. (2000) FEBS Lett 476, 198-202.

1. A polynucleotide encoding a functional Type II gonadotropin-releasing hormone receptor (Type II GnRH-R) peptide.

2. A polynucleotide as claimed in claim 1 encoding at least a portion of exon I of Type II GnRH-R.

3. A polynucleotide as claimed in claim 1 which encodes mammalian Type II GnRH-R.

4. A polynucleotide as claimed in claim 3 which encodes a primate Type II GnRH-R.

5. A polynucleotide as claimed in claim 4 which encodes marmoset Type II GnRH-R.

6. A polynucleotide as claimed in claim 5 having the nucleotide sequence as set out in SEQ ID No. 1 or which has over 90% homology thereto.

7. A polynucleotide as claimed in claim 4 which encodes human Type II GnRH-R.

8. A polynucleotide as claimed in claim 7 which comprises a nucleotide sequence as set out in any one of SEQ ID Nos. 3, 5, 7, 9, 36, 38, 40 or 42, or which has over 90% homology thereto.

9. A polynucleotide is claimed in claim 8 which encodes an amino acid sequence as set out in any one of SEQ ID Nos. 4, 6, 8, 10, 37, 39, 41 or 43, or which has over 90% homology thereto.

10. A polynucleotide as claimed in claim 1 which encodes a peptide able to bind specifically to Type II GnRH.

11. A polynucleotide as claimed in claim 10 which encodes a peptide able to act as a receptor for Type II GnRH-R.

12. A recombinant genetic construct comprising a polynucleotide as claimed in claim 1.

13. An expression vector comprising a polynucleotide as claimed in claim 1 and able to express functional Type II GnRH-R peptide.

14. A host cell transformed with a vector as claimed in claim 13.

15. A host cell as claimed in claim 14 able to express functional Type II GnRH-R.

16. A transgenic animal having a construct as claimed in claim 12 stably integrated into its genome.

17. An isolated functional Type II gonadotropin-releasing hormone receptor (Type II GnRH-R).

18. A peptide comprising at least a portion of exon I of Type II gonadotropin-releasing hormone receptor (Type II GnRH-R).

19. A peptide as claimed in claim 17 which comprises a portion of exon I of mammalian Type II GnRH-R.

20. A peptide as claimed in claim 19 which comprises a portion of exon I of primate Type II GnRH-R.

21. A peptide as claimed in claim 17 having an amino acid sequence as set out in SEQ ID No. 2 which has over 90% homology thereto.

22. A peptide as claimed in claim 17 having an amino acid sequence as set out in any one of SEQ ID Nos. 4, 6, 8, 10, 37, 39, 41 or 43, or which has over 90% homology thereto.

23. A peptide as claimed in claim 17 which is able to bind Type II GnRH specifically.

24. A peptide as claimed in claim 23 which is a functional receptor for Type II GnRH.

25. An antibody able to bind specifically to Type II GnRH-R.

26. An antibody as claimed in claim 25 which is specific to an extracellular domain EC1, EC2 or EC3 of Type II GnRH-R.

27. A method of screening an agent for pharmacological activity, said method comprising:
a) providing functional Type II GnRH-R peptide and exposing said peptide to the agent; and
b) ascertaining whether said agent interacts with said Type II GnRH-R peptide.

28. The method as claimed in claim 27 wherein said Type II GnRH-R is expressed by a host cell as claimed in claim 15.

29. The method as claimed in claim 27 wherein said Type II GnRH-R is expressed by a host cell transformed with a polynucleotide as claimed in claim 11 wherein said host cell imitates Type II GnRH-R signal transduction at least partially and wherein exposure of said host cell to said agent results in Type II GnRH-R signal transduction when said agent binds successfully to said Type II GnRH-R peptide.

30. A method of inhibiting binding of GnRH to its native receptor in vivo, said method comprising administering Type II GnRH-R or an extracellular domain thereof.

31. The method of claim 30 wherein the EC2 loop of Type II GnRH-R is administered.

32. A method of contraception, said method comprising administering exogenous Type II GnRH-R or an extracellular domain thereof to a patient in quantities sufficient to substantially diminish binding of endogenous Type II GnRH to endogenous Type II GnRH-R.


SEQUENCE LISTING (43 sequences)

SEQ ID NO: 1 - DNA, 1465 bp, Callithrix jacchus
  CDS (41)..(1180)
acttcatactcacacttcatcctcctatctccaggccaccatgtctgcagtaaacggcaccccttgggggtcctcagcgcgggaggaggtctgggcaggatcgggagtggaggtggagggctcagagctgcccaccttctcgacagcagcaaaggtccgagtgggagtgaccattgtgctgtttgtttcttcggctggagggaacctggctgtcctgtggtcagtgacacggccgcaacccagccagctccgcccctctccggtcaggagactcttcgcccatttagcagccgccgacttactagtcacttttgtggttatgcccctagatgccacctggaatatcactgttcagtggctggctggggacatcgcatgtcggacactcatgttcctgaaactaatggccatgtatgctgcagctttcctgcctgtggtcattggactggatcgccaggcagcagtactcaatccgcttggatcccgctcaggtgtaaggaaacttctgggggcagcctggggacttagtttcctgcttgccttgccccagctgttcctgttccataccgtccaccgagctggtccagttcccttcactcagtgtgccaccaaagggagcttcaaggctcgatggcaagagaccacctataacctcttcactttctgctgcctctttctgctgccactgactgccatggccatctgctatagccgcattgtgctcggtgtgtccagcccccggacaaggaaggggagccatgcccctgccggggaatttgccctccgtcgctccttcgacaatcgtccccgtgtccgtcttcgggccctgagactggccctgctcgtcttgctgaccttcatcctctgctggacaccttattacttactaggtctgtggtactggttttccccgagcatgctaagtgaagtccctcccagcctcagccacatccttttcctctttggcctcctcaatgctcctttggatcctctcctctatggggccttcacccttggctgccgaagagggcaccaagaacttagtatggactcttctagggaagaagggtctaggagaatgttccaacaggacattcaggcccttagacaaacggaggtacaaaaaactgtgacatcaagaaaggcaggagaaacaaaagacattcctataacatcaatctgatcctaacacagtatagagaaacacaataattctttaataccataagatcttaacgtctcaatttcctgctctcctaatcccccccaaaagaaatactgagcagtgtctccatttaaaccctgcctgaacttgagactatgtctaatacagaaactcacacaactagcctgggcaacacagtgagacctaatctctatagaaatattaaaaggttaagccaggcatggtggcatgtgcctggaaccccagctactgggagggtgaggcagaaggatggcttgg

SEQ ID NO: 2 - protein, 380 aa, Callithrix jacchus
MSAVNGTPWGSSAREEVWAGSGVEVEGSELPTFSTAAKVRVGVTIVLFVSSAGGNLAVLWSVTRPQPSQLRPSPVRRLFAHLAAADLLVTFVVMPLDATWNITVQWLAGDIACRTLMFLKLMAMYAAAFLPVVIGLDRQAAVLNPLGSRSGVRKLLGAAWGLSFLLALPQLFLFHTVHRAGPVPFTQCATKGSFKARWQETTYNLFTFCCLFLLPLTAMAICYSRIVLGVSSPRTRKGSHAPAGEFALRRSFDNRPRVRLRALRLALLVLLTFILCWTPYYLLGLWYWFSPSMLSEVPPSLSHILFLFGLLNAPLDPLLYGAFTLGCRRGHQELSMDSSREEGSRRMFQQDIQALRQTEVQKTVTSRKAGETKDIPITSI

SEQ ID NO: 3 - DNA, 2650 bp, Homo sapiens
  misc_feature (292)..(292): n is absent in the human sequence, but included herein for ease of alignment with the corresponding marmoset sequence.
  misc_feature (292)..(297): 292-297 is a possible intron
  misc_feature (292)..(300): 292-300 is a possible intron
  misc_feature (292)..(330): 292-330 is a possible intron
  misc_feature (798)..(800): 798-800 is the codon tga translated as a selenocysteine (amino acid No. 179)
  CDS (264)..(1400): The CDS shown includes some apparent amino acids (from Gly 10 onwards) which would be deleted in a short intron.
cagtagaggcctgaagcccaggctggtctgtccaaggaaaaaggagcgtgattggtaccagatcttcgttccctgcagaaccttgacagttgaacaagtgacctcctccagaacagatggagagtctccagaagcagaggctttagtgaacgaaattcgcaataatcagctccagatcctgaaaaggagggcgaagaatcagtggccaaagctaaccgcttcatacccacacttcatcctcctcagtttctctccaggccaccatgtctgcaggcaacggcaccccttggggntcagcagcgggggaggaggtctgggctggatcaggagtggaggtggagggctcagagctgcccaccttctcggcagcagccaaggtccgagtgggagtgaccattgtgctgtttgtttcttcggctggagggaacctggcagtcctgtggtcagtgacacggcgggaacccagccagctccgcccctgtccggtcaggagactcttcatccatttagcagccgccgacttactagtcacttttgtggttatgcccctagatgccacctggaatatcactgttcaatggctggctgtggacatcgcatgtcggacactgatgttcctgaaactaatggccacgtattctgcagctttcctgcctgtggtcattggattggaccgccaggcagcagtactcaacccgcttggatcccgttcaggtgtaaggaaacttctgggggcagcctggggacttagtttcctgcttgccttcccccagctgttcctgttccacacggtccacnnngctggcccagtccctttcactcagtgtgtcaccaaaggcagcttcaaggctcaatggcaagagaccacctataacctcttcaccttctgctgcctccttctgctgccactgactgccatggccatctgctatagccgcattgtcctcagtgtgtccaggccccagacaaggaaggggagccatgcccctgctggtgaatttgccctcccccgctcctttgacaattgtccccgtgttcgtctccgggccctgagactggccctgcttaacttactgaccttcatcctctgctggacaccttattacctactgggtatgtggtactggttctcccccaccatgctaactgaagtccctcccagcctgagccacatccttttcctcttgggcctcctcaatgctcctttggatcctctcctctatggggccttcacccttggctgccgaagagggcaccaagaacttagtatagactcttctaaagaagggtctgggagaatgctccaagaggagattcatgcctttagacagctggaagtacaaaaaactgtgacatcaagaagggcaggagaaacaaaaggcatttctataacatctatctgatcctaacagagtatgtaggaacagaatagtaagtctttagtgccataagatcttaacatctcacttctactcctgctctcctagttccccccaaaaaagaaatactgaccagtgtctctactttaaaccctacctgaaacttgagactatgtctaatatagaaactcacataactagcccaggtaacacagcaagaccccatctctacaaaaatattaaaaatttagctgggcatggtggcatgtgcctgtaatcccaactactagggacagtgaggcagaaggatggcttgagcccaagagtttgaagctgcagtgagctatgatcagctgcaatccaccctgggtaacacagcaagactctatctcaaaaaaaagaaaaaaaagaaatacatagagttcagtccctagaagtatcttcacaatgatccatacagccttgctatgctttagaactttcaattttaggacaggaaagtaacattaaatgtagaaaacaaaaatggaacatttattcgcaactcaaatactacgcatatacggtaagagattaaatataaacacagcaagttccaccccagtcctatttgtccaaggctgcatggtcaaatggaatcttgaagagaacacctggacaacagaggacctgtcagcgacgtctccggtctggacttctgctgcgtcttcggccacctctagggaaaaagaagcagggagaggagtccattagagggacataatactaagtcctcattctgtttcgttcgtattcccttcacccagacagtatttgccctcttcattttacctcctcttgccttttggtggaccccgaacaaaacaccagtcaacgctgatgggctgtcccatcaaatcctggccattgagtccctccatagcagcctgggcttccttgtatgtttcatattcaactagagtatacccctggggaaagtgaaaagacagatatgaaaaccctcctatttccccaagtatcactgagtatctttttcctcaaatctaaactcagaaaaatctacacactttaaacaatgaatgtacatcatatatctctactgtatatatacatcatctccgtttctgtctctttggcgtgtctgacaaaacatagatttccaatgtcattttatttcaactttgctcttggccaaccacagcaaacacagaactacgaatgctacctaaggcttatgaaagaaaggaggcaataaagtgtcacttaggata

SEQ ID NO: 4 - protein, 379 aa, Homo sapiens
  misc_feature (179)..(179)
  misc_feature (292)..(292): n is absent in the human sequence, but included herein for ease of alignment with the corresponding marmoset sequence.
  misc_feature (292)..(297): 292-297 is a possible intron
  misc_feature (292)..(300): 292-300 is a possible intron
  misc_feature (292)..(330): 292-330 is a possible intron
  misc_feature (798)..(800): 798-800 is the codon tga translated as a selenocysteine (amino acid No. 179)
MSAGNGTPWGSAAGEEVWAGSGVEVEGSELPTFSAAAKVRVGVTIVLFVSSAGGNLAVLWSVTRREPSQLRPCPVRRLFIHLAAADLLVTFVVMPLDATWNITVQWLAVDIACRTLMFLKLMATYSAAFLPVVIGLDRQAAVLNPLGSRSGVRKLLGAAWGLSFLLAFPQLFLFHTVHXAGPVPFTQCVTKGSFKAQWQETTYNLFTFCCLLLLPLTAMAICYSRIVLSVSRPQTRKGSHAPAGEFALPRSFDNCPRVRLRALRLALLNLLTFILCWTPYYLLGMWYWFSPTMLTEVPPSLSHILFLLGLLNAPLDPLLYGAFTLGCRRGHQELSIDSSKEGSGRMLQEEIHAFRQLEVQKTVTSRRAGETKGISITSI

SEQ ID NO: 5 - DNA, 1397 bp, Homo sapiens
  CDS (264)..(1394)
  misc_feature (792)..(794): 792-794 is the codon tga translated as selenocysteine (amino acid No. 177)
cagtagaggcctgaagcccaggctggtctgtccaaggaaaaaggagcgtgattggtaccagatcttcgttccctgcagaaccttgacagttgaacaagtgacctcctccagaacagatggagagtctccagaagcagaggctttagtgaacgaaattcgcaataatcagctccagatcctgaaaaggagggcgaagaatcagtggccaaagctaaccgcttcatacccacacttcatcctcctcagtttctctccaggccaccatgtctgcaggcaacggcaccccttgggcagcgggggaggaggtctgggctggatcaggagtggaggtggagggctcagagctgcccaccttctcggcagcagccaaggtccgagtgggagtgaccattgtgctgtttgtttcttcggctggagggaacctggcagtcctgtggtcagtgacacggcgggaacccagccagctccgcccctgtccggtcaggagactcttcatccatttagcagccgccgacttactagtcacttttgtggttatgcccctagatgccacctggaatatcactgttcaatggctggctgtggacatcgcatgtcggacactgatgttcctgaaactaatggccacgtattctgcagctttcctgcctgtggtcattggattggaccgccaggcagcagtactcaacccgcttggatcccgttcaggtgtaaggaaacttctgggggcagcctggggacttagtttcctgcttgccttcccccagctgttcctgttccacacggtccacnnngctggcccagtccctttcactcagtgtgtcaccaaaggcagcttcaaggctcaatggcaagagaccacctataacctcttcaccttctgctgcctccttctgctgccactgactgccatggccatctgctatagccgcattgtcctcagtgtgtccaggccccagacaaggaaggggagccatgcccctgctggtgaatttgccctcccccgctcctttgacaattgtccccgtgttcgtctccgggccctgagactggccctgcttaacttactgaccttcatcctctgctggacaccttattacctactgggtatgtggtactggttctcccccaccatgctaactgaagtccctcccagcctgagccacatccttttcctcttgggcctcctcaatgctcctttggatcctctcctctatggggccttcacccttggctgccgaagagggcaccaagaacttagtatagactcttctaaagaagggtctgggagaatgctccaagaggagattcatgcctttagacagctggaagtacaaaaaactgtgacatcaagaagggcaggagaaacaaaaggcatttctataacatctatctga

SEQ ID NO: 6 - protein, 377 aa, Homo sapiens
  misc_feature (792)..(794): 792-794 is the codon tga translated as selenocysteine (amino acid No. 177)
MSAGNGTPWAAGEEVWAGSGVEVEGSELPTFSAAAKVRVGVTIVLFVSSAGGNLAVLWSVTRREPSQLRPCPVRRLFIHLAAADLLVTFVVMPLDATWNITVQWLAVDIACRTLMFLKLMATYSAAFLPVVIGLDRQAAVLNPLGSRSGVRKLLGAAWGLSFLLAFPQLFLFHTVHXAGPVPFTQCVTKGSFKAQWQETTYNLFTFCCLLLLPLTAMAICYSRIVLSVSRPQTRKGSHAPAGEFALPRSFDNCPRVRLRALRLALLNLLTFILCWTPYYLLGMWYWFSPTMLTEVPPSLSHILFLLGLLNAPLDPLLYGAFTLGCRRGHQELSIDSSKEGSGRMLQEEIHAFRQLEVQKTVTSRRAGETKGISITSI

SEQ ID NO: 7 - DNA, 1394 bp, Homo sapiens
  CDS (264)..(1391)
  misc_feature (789)..(791): codon 789-791 is the codon tga translated as a selenocysteine (amino acid No. 176).
cagtagaggcctgaagcccaggctggtctgtccaaggaaaaaggagcgtgattggtaccagatcttcgttccctgcagaaccttgacagttgaacaagtgacctcctccagaacagatggagagtctccagaagcagaggctttagtgaacgaaattcgcaataatcagctccagatcctgaaaaggagggcgaagaatcagtggccaaagctaaccgcttcatacccacacttcatcctcctcagtttctctccaggccaccatgtctgcaggcaacggcaccccttgggcgggggaggaggtctgggctggatcaggagtggaggtggagggctcagagctgcccaccttctcggcagcagccaaggtccgagtgggagtgaccattgtgctgtttgtttcttcggctggagggaacctggcagtcctgtggtcagtgacacggcgggaacccagccagctccgcccctgtccggtcaggagactcttcatccatttagcagccgccgacttactagtcacttttgtggttatgcccctagatgccacctggaatatcactgttcaatggctggctgtggacatcgcatgtcggacactgatgttcctgaaactaatggccacgtattctgcagctttcctgcctgtggtcattggattggaccgccaggcagcagtactcaacccgcttggatcccgttcaggtgtaaggaaacttctgggggcagcctggggacttagtttcctgcttgccttcccccagctgttcctgttccacacggtccacnnngctggcccagtccctttcactcagtgtgtcaccaaaggcagcttcaaggctcaatggcaagagaccacctataacctcttcaccttctgctgcctccttctgctgccactgactgccatggccatctgctatagccgcattgtcctcagtgtgtccaggccccagacaaggaaggggagccatgcccctgctggtgaatttgccctcccccgctcctttgacaattgtccccgtgttcgtctccgggccctgagactggccctgcttaacttactgaccttcatcctctgctggacaccttattacctactgggtatgtggtactggttctcccccaccatgctaactgaagtccctcccagcctgagccacatccttttcctcttgggcctcctcaatgctcctttggatcctctcctctatggggccttcacccttggctgccgaagagggcaccaagaacttagtatagactcttctaaagaagggtctgggagaatgctccaagaggagattcatgcctttagacagctggaagtacaaaaaactgtgacatcaagaagggcaggagaaacaaaaggcatttctataacatctatctga

SEQ ID NO: 8 - protein, 376 aa, Homo sapiens
  misc_feature (789)..(791): codon 789-791 is the codon tga translated as a selenocysteine (amino acid No. 176).
MSAGNGTPWAGEEVWAGSGVEVEGSELPTFSAAAKVRVGVTIVLFVSSAGGNLAVLWSVTRREPSQLRPCPVRRLFIHLAAADLLVTFVVMPLDATWNITVQWLAVDIACRTLMFLKLMATYSAAFLPVVIGLDRQAAVLNPLGSRSGVRKLLGAAWGLSFLLAFPQLFLFHTVHXAGPVPFTQCVTKGSFKAQWQETTYNLFTFCCLLLLPLTAMAICYSRIVLSVSRPQTRKGSHAPAGEFALPRSFDNCPRVRLRALRLALLNLLTFILCWTPYYLLGMWYWFSPTMLTEVPPSLSHILFLLGLLNAPLDPLLYGAFTLGCRRGHQELSIDSSKEGSGRMLQEEIHAFRQLEVQKTVTSRRAGETKGISITSI

SEQ ID NO: 9 - DNA, 1364 bp, Homo sapiens
  CDS (264)..(1361)
  misc_feature (759)..(761): codon 759-761 is the codon tga translated as selenocysteine (amino acid 166)
cagtagaggcctgaagcccaggctggtctgtccaaggaaaaaggagcgtgattggtaccagatcttcgttccctgcagaaccttgacagttgaacaagtgacctcctccagaacagatggagagtctccagaagcagaggctttagtgaacgaaattcgcaataatcagctccagatcctgaaaaggagggcgaagaatcagtggccaaagctaaccgcttcatacccacacttcatcctcctcagtttctctccaggccaccatgtctgcaggcaacggcaccccttgggtggaggtggagggctcagagctgcccaccttctcggcagcagccaaggtccgagtgggagtgaccattgtgctgtttgtttcttcggctggagggaacctggcagtcctgtggtcagtgacacggcgggaacccagccagctccgcccctgtccggtcaggagactcttcatccatttagcagccgccgacttactagtcacttttgtggttatgcccctagatgccacctggaatatcactgttcaatggctggctgtggacatcgcatgtcggacactgatgttcctgaaactaatggccacgtattctgcagctttcctgcctgtggtcattggattggaccgccaggcagcagtactcaacccgcttggatcccgttcaggtgtaaggaaacttctgggggcagcctggggacttagtttcctgcttgccttcccccagctgttcctgttccacacggtccacnnngctggcccagtccctttcactcagtgtgtcaccaaaggcagcttcaaggctcaatggcaagagaccacctataacctcttcaccttctgctgcctccttctgctgccactgactgccatggccatctgctatagccgcattgtcctcagtgtgtccaggccccagacaaggaaggggagccatgcccctgctggtgaatttgccctcccccgctcctttgacaattgtccccgtgttcgtctccgggccctgagactggccctgcttaacttactgaccttcatcctctgctggacaccttattacctactgggtatgtggtactggttctcccccaccatgctaactgaagtccctcccagcctgagccacatccttttcctcttgggcctcctcaatgctcctttggatcctctcctctatggggccttcacccttggctgccgaagagggcaccaagaacttagtatagactcttctaaagaagggtctgggagaatgctccaagaggagattcatgcctttagacagctggaagtacaaaaaactgtgacatcaagaagggcaggagaaacaaaaggcatttctataacatctatctga

SEQ ID NO: 10 - protein, 366 aa, Homo sapiens
  misc_feature (759)..(761): codon 759-761 is the codon tga translated as selenocysteine (amino acid 166)
MSAGNGTPWVEVEGSELPTFSAAAKVRVGVTIVLFVSSAGGNLAVLWSVTRREPSQLRPCPVRRLFIHLAAADLLVTFVVMPLDATWNITVQWLAVDIACRTLMFLKLMATYSAAFLPVVIGLDRQAAVLNPLGSRSGVRKLLGAAWGLSFLLAFPQLFLFHTVHXAGPVPFTQCVTKGSFKAQWQETTYNLFTFCCLLLLPLTAMAICYSRIVLSVSRPQTRKGSHAPAGEFALPRSFDNCPRVRLRALRLALLNLLTFILCWTPYYLLGMWYWFSPTMLTEVPPSLSHILFLLGLLNAPLDPLLYGAFTLGCRRGHQELSIDSSKEGSGRMLQEEIHAFRQLEVQKTVTSRRAGETKGISITSI

SEQ ID NO: 11 - DNA, 51 bp, Homo sapiens
  CDS (1)..(51)
atgtctgcaggcaacggcaccccttgggcagcgggggaggaggtctgggct

SEQ ID NO: 12 - protein, 17 aa, Homo sapiens
MSAGNGTPWAAGEEVWA

SEQ ID NO: 13 - DNA, 51 bp, Homo sapiens
  CDS (1)..(51)
atgtctgcaggcaacggcaccccttgggcgggggaggaggtctgggctgga

SEQ ID NO: 14 - protein, 17 aa, Homo sapiens
MSAGNGTPWAGEEVWAG

SEQ ID NO: 15 - DNA, 51 bp, Homo sapiens
  CDS (1)..(51)
atgtctgcaggcaacggcaccccttgggtggaggtggagggctcagagctg

SEQ ID NO: 16 - protein, 17 aa, Homo sapiens
MSAGNGTPWVEVEGSEL

SEQ ID NO: 17 - DNA, 22 bp, Synthetic primer
  misc_feature: Synthetic primer directed to the Type II marmoset (Callithrix jac chus) GnRH receptor exon sequences
gatgccacctggaatatcactg

SEQ ID NO: 18 - DNA, 13 bp, synthetic primer
  misc_feature: Synthetic primer directed to the Type II marmoset (callithrix Jacchus) GnRH receptor exon sequences
aggcagcagaagg

SEQ ID NO: 19 - DNA, 23 bp, synthetic primer
  misc_feature: Synthetic primer directed to the Type II marmoset (Callithrix jacchus) GnRH receptor exon sequences
cagcctggggacttagtttcctg

SEQ ID NO: 20 - DNA, 20 bp, Synthetic primer
  misc_feature: Synthetic primer directed to the Type II marmoset ( Callithrix jacchus) GnRH receptor exon sequences.
ggttataggtggtctcttgc

SEQ ID NO: 21 - DNA, 39 bp, synthetic primer
  misc_feature: Synthetic primer directed to the type II marmoset (Callithrix jacchus) GnRH receptor exon sequences.
gaccacgcgtatcgatgtcgacttttttttttttttttv

SEQ ID NO: 22 - DNA, 20 bp, synthetic primer
  misc_feature: Synthetic primer directed to the Type II marmoset (Callithrix jacchus) GnRH receptor exon sequences
gaagggactggaccagctcg

SEQ ID NO: 23 - DNA, 22 bp, synthetic primer
  misc_feature: Synthetic primer directed to the Type II marmoset (Callithrix jacchus) GnRH receptor exon sequences.
gaccacgcgtatcgatgtcgac

SEQ ID NO: 24 - DNA, 21 bp, Synthetic primer
  misc_feature: Synthetic primer directed to the Type II marmoset (Callithrix jacchus) GnRH receptor exon sequences.
caaggcaagcaggaaactaag

SEQ ID NO: 25 - DNA, 22 bp, Synthetic primer
  misc_feature: Synthetic primer directed to the Type II marmoset (Callithrix jacchus) GnRH receptor exon sequences.
acctcttcaccttctgctgcct

SEQ ID NO: 26 - DNA, 23 bp, Synthetic primer
  misc_feature: Synthetic primer directed to the Type II marmoset (Callithrix jacchus) GnRH receptor exon sequences.
cctcctcaatgctcctttggatc

SEQ ID NO: 27 - DNA, 27 bp, Synthetic primer
  misc_feature: Synthetic primer directed to the Type II marmoset (Callithrix jacchus) GnRH receptor exon sequences.
gaattcgcttcatactcacacttcatc

SEQ ID NO: 28 - DNA, 30 bp, Synthetic primer
  misc_feature: Synthetic primer directed to the Type II marmoset (Callithrix jacchus) GnRH receptor exon sequences.
cggaattctcacacttcatcctcctatctc

SEQ ID NO: 29 - DNA, 32 bp, Synthetic primer
  misc_feature: Synthetic primer directed to the Type II marmoset (Callithrix jacchus) GnRH receptor exon sequences.
gctctagagatcagattgatgttataggaatg

SEQ ID NO: 30 - protein, 12 aa, synthetic peptide
  misc_feature: Synthetic peptide corresponding to EC3 of human (Homo sapiens) Type II GnRH receptor.
YSPTMLTEVPPC

SEQ ID NO: 31 - protein, 12 aa, synthetic peptide
  misc_feature: Synthetic peptide to EC3 of the human (Homo sapiens) Type I GnRH receptor
DPEMLNRLSDPC

SEQ ID NO: 32 - DNA, 20 bp, Synthetic primer
  misc_feature: Synthetic primer (sense) : Marmoset (Callithrix jacchus) Type II GnRH receptor.
cttcggctggagggaacctg

SEQ ID NO: 33 - DNA, 18 bp, Synthetic primer
  misc_feature: Synthetic primer (anti sense) : Marmoset (Callithrix jacchus) Type II GnRH receptor
ggtgccctcttcggcagc

SEQ ID NO: 34 - DNA, 20 bp, Synthetic primer
  misc_feature: Synthetic primer (sense) : Human (Home sapiens) Type II GnRH receptor
ctgtcctgcccggtcctgag

SEQ ID NO: 35 - DNA, 20 bp, Synthetic primer
  misc_feature: Synthetic primer (antisense) : Human (Homo sapiens) Type II GnRH receptor
tgcccaccttctcggcagca

SEQ ID NO: 36 - DNA, 2650 bp, Homo sapiens
  misc_feature (292)..(292)223> OTHER INFORMATION: n is absent in the human sequence, but includedherein for ease of alignment with the corresponding marmosetsequence.
  misc_feature (292)..(297): 292-297 is a possible intron
  misc_feature (292)..(300): 292-300 is a possible intron
  misc_feature (292)..(330): 292-330 is a possible intron
  misc_feature (798)..(800): 798-800 is the codon tga translated as a seleno cysteine (amino acid No. 179)
  CDS (264)..(1400): The CDS shown includes some apparent amino acids (from Gly 10 onwards) which would be deleted in a short intron.
cagtagaggcctgaagcccaggctggtctgtccaaggaaaaaggagcgtgattggtaccagatcttcgttccctgcagaaccttgacagttgaacaagtgacctcctccagaacagatggagagtctccagaagcagaggctttagtgaacgaaattcgcaataatcagctccagatcctgaaaaggagggcgaagaatcagtggccaaagctaaccgcttcatacccacacttcatcctcctcagtttctctccaggccaccatgtctgcaggcaacggcaccccttggggntcagcagcgggggaggaggtctgggctggatcaggagtggaggtggagggctcagagctgcccaccttctcggcagcagccaaggtccgagtgggagtgaccattgtgctgtttgtttcttcggctggagggaacctggcagtcctgtggtcagtgacacggcgggaacccagccagctccgcccctstccggtcaggagactcttcatccatttagcagccgccgacttactagtcacttttgtggttatgcccctagatgccacctggaatatcactgttcaatggctggctgtggacatcgcatgtcggacactgatgttcctgaaactaatggccacgtattctgcagctttcctgcctgtggtcattggattggaccgccaggcagcagtactcaacccgcttggatcccgttcaggtgtaaggaaacttctgggggcagcctggggacttagtttcctgcttgccttcccccagctgttcctgttccacacggtccacnnngctggcccagtccctttcactcagtgtgtcaccaaaggcagcttcaaggctcaatggcaagagaccacctataacctcttcaccttctgctgcctcyttctgctgccactgactgccatggccatctgctatagccgcattgtcctcagtgtgtccaggccccagacaaggaaggggagccatgcccctgctggtgaatttgccctcccccgctcctttgacaattgtccccgtgttcgtctccgggccctgagactggccctgcttatcttgctgaccttcatcctctgctggacaccttattacctactgggtatgtggtactggttctcccccaccatgctaactgaagtccctcccagcctgagccacatccttttcctcttgggcctcctcaatgctcctttggatcctctcctctatggggccttcacccttggctgccgaagagggcaccaagaacttagtatagactcttctaaagaagggtctgggagaatgctccaagaggagattcatgcctttagacagctggaagtacaaaaaactgtgacatcaagaagggcaggagaaacaaaaggcatttctataacatctatctgatcctaacagagtatgtaggaacagaatagtaagtctttagtgccataagatcttaacatctcacttctactcctgctctcctagttccccccaaaaaagaaatactgaccagtgtctctactttaaaccctacctgaaacttgagactatgtctaatatagaaactcacataactagcccaggtaacacagcaagaccccatctctacaaaaatattaaaaatttagctgggcatggtggcatgtgcctgtaatcccaactactagggacagtgaggcagaaggatggcttgagcccaagagtttgaagctgcagtgagctatgatcagctgcaatccaccctgggtaacacagcaagactctatctcaaaaaaaagaaaaaaaagaaatacatagagttcagtccctagaagtatcttcacaatgatccatacagccttgctatgctttagaactttcaattttaggacaggaaagtaacattaaatgtagaaaacaaaaatggaacatttattcgcaactcaaatactacgcatatacggtaagagattaaatataaacacagcaagttccaccccagtcctatttgtccaaggctgcatggtcaaatggaatcttgaagagaacacctggacaacagaggacctgtcagcgacgtctccggtctggacttctgctgcgtcttcggccacctctagggaaaaagaagcagggagaggagtccattagagggacataatactaagtcctcattctgtttcgttcgtattcccttcacccagacagtatttgccctcttcattttacctcctcttgccttttggtggaccccgaacaaaacaccagtcaacgctgatgggctgtcccatcaaatcctggccattgagtccctccatagcagcctgggcttccttgtatgtttcatattcaactagagtatacccctggggaaagtgaaaagacagatatgaaaaccctcctatttccccaagtatcactgagtatctttttcctcaaatctaaactcagaaaaatctacacactttaaacaatgaatgtacatcatatatctctactgtatatatacatcatctccgtttctgtctctttggcgtgtctgacaaaacatagatttccaatgtcattttatttcaactttgctcttggccaaccacagcaaacacagaactacgaatgctacctaaggcttatgaaagaaaggaggcaataaagtgtcacttaggata

SEQ ID NO: 37 - protein, 379 aa, Homo sapiens
  misc_feature (73)..(73): The ′Xaa′ at location 73 stands for Cys, or Ser.
  misc_feature (212)..(212): The ′Xaa′ at location 212 stands for Leu, or Phe.
  misc_feature (292)..(292): n is absent in the human sequence, but included herein for ease of alignment with the corresponding marmoset sequence.
  misc_feature (292)..(297): 292-297 is a possible intron
  misc_feature (292)..(300): 292-300 is a possible intron
  misc_feature (292)..(330): 292-330 is a possible intron
  misc_feature (798)..(800): 798-800 is the codon tga translated as a seleno cysteine (amino acid No. 179)
MSAGNGTPWGSAAGEEVWAGSGVEVEGSELPTFSAAAKVRVGVTIVLFVSSAGGNLAVLWSVTRREPSQLRPXPVRRLFIHLAAADLLVTFVVMPLDATWNITVQWLAVDIACRTLMFLKLMATYSAAFLPVVIGLDRQAAVLNPLGSRSGVRKLLGAAWGLSFLLAFPQLFLFHTVHXAGPVPFTQCVTKGSFKAQWQETTYNLFTFCCLXLLPLTAMAICYSRIVLSVSRPQTRKGSHAPAGEFALPRSFDNCPRVRLRALRLALLILLTFILCWTPYYLLGMWYWFSPTMLTEVPPSLSHILFLLGLLNAPLDPLLYGAFTLGCRRGHQELSIDSSKEGSGRMLQEEIHAFRQLEVQKTVTSRRAGETKGISITSI

SEQ ID NO: 38 - DNA, 1397 bp, Homo sapiens
  CDS (264)..(1394)
  misc_feature (792)..(794): codon 792-794 is the codon tga translated as selenocysteine (amino acid 166)
cagtagaggcctgaagcccaggctggtctgtccaaggaaaaaggagcgtgattggtaccagatcttcgttccctgcagaaccttgacagttgaacaagtgacctcctccagaacagatggagagtctccagaagcagaggctttagtgaacgaaattcgcaataatcagctccagatcctgaaaaggagggcgaagaatcagtggccaaagctaaccgcttcatacccacacttcatcctcctcagtttctctccaggccaccatgtctgcaggcaacggcaccccttgggcagcgggggaggaggtctgggctggatcaggagtggaggtggagggctcagagctgcccaccttctcggcagcagccaaggtccgagtgggagtgaccattgtgctgtttgtttcttcggctggagggaacctggcagtcctgtggtcagtgacacggcgggaacccagccagctccgcccctstccggtcaggagactcttcatccatttagcagccgccgacttactagtcacttttgtggttatgcccctagatgccacctggaatatcactgttcaatggctggctgtggacatcgcatgtcggacactgatgttcctgaaactaatggccacgtattctgcagctttcctgcctgtggtcattggattggaccgccaggcagcagtactcaacccgcttggatcccgttcaggtgtaaggaaacttctgggggcagcctggggacttagtttcctgcttgccttcccccagctgttcctgttccacacggtccacnnngctggcccagtccctttcactcagtgtgtcaccaaaggcagcttcaaggctcaatggcaagagaccacctataacctcttcaccttctgctgcctcyttctgctgccactgactgccatggccatctgctatagccgcattgtcctcagtgtgtccaggccccagacaaggaaggggagccatgcccctgctggtgaatttgccctcccccgctcctttgacaattgtccccgtgttcgtctccgggccctgagactggccctgcttatcttgctgaccttcatcctctgctggacaccttattacctactgggtatgtggtactggttctcccccaccatgctaactgaagtccctcccagcctgagccacatccttttcctcttgggcctcctcaatgctcctttggatcctctcctctatggggccttcacccttggctgccgaagagggcaccaagaacttagtatagactcttctaaagaagggtctgggagaatgctccaagaggagattcatgcctttagacagctggaagtacaaaaaactgtgacatcaagaagggcaggagaaacaaaaggcatttctataacatctatctga

SEQ ID NO: 39 - protein, 377 aa, Homo sapiens
  misc_feature (71)..(71): The ′Xaa′ at location 71 stands for Cys, or Ser.
  misc_feature (210)..(210): The ′Xaa′ at location 210 stands for Leu, or Phe.
  misc_feature (792)..(794): codon 792-794 is the codon tga translated as selenocysteine (amino acid 166)
MSAGNGTPWAAGEEVWAGSGVEVEGSELPTFSAAAKVRVGVTIVLFVSSAGGNLAVLWSVTRREPSQLRPXPVRRLFIHLAAADLLVTFVVMPLDATWNITVQWLAVDIACRTLMFLKLMATYSAAFLPVVIGLDRQAAVLNPLGSRSGVRKLLGAAWGLSFLLAFPQLFLFHTVHXAGPVPFTQCVTKGSFKAQWQETTYNLFTFCCLXLLPLTAMAICYSRIVLSVSRPQTRKGSHAPAGEFALPRSFDNCPRVRLRALRLALLILLTFILCWTPYYLLGMWYWFSPTMLTEVPPSLSHILFLLGLLNAPLDPLLYGAFTLGCRRGHQELSIDSSKEGSGRMLQEEIHAFRQLEVQKTVTSRRAGETKGISITSI

SEQ ID NO: 40 - DNA, 1394 bp, Homo sapiens
  CDS (264)..(1391)
  misc_feature (789)..(791): codon 789-791 is the codon tga translated as selenocysteine (amino acid 166)
cagtagaggcctgaagcccaggctggtctgtccaaggaaaaaggagcgtgattggtaccagatcttcgttccctgcagaaccttgacagttgaacaagtgacctcctccagaacagatggagagtctccagaagcagaggctttagtgaacgaaattcgcaataatcagctccagatcctgaaaaggagggcgaagaatcagtggccaaagctaaccgcttcatacccacacttcatcctcctcagtttctctccaggccaccatgtctgcaggcaacggcaccccttgggcgggggaggaggtctgggctggatcaggagtggaggtggagggctcagagctgcccaccttctcggcagcagccaaggtccgagtgggagtgaccattgtgctgtttgtttcttcggctggagggaacctggcagtcctgtggtcagtgacacggcgggaacccagccagctccgcccctstccggtcaggagactcttcatccatttagcagccgccgacttactagtcacttttgtggttatgcccctagatgccacctggaatatcactgttcaatggctggctgtggacatcgcatgtcggacactgatgttcctgaaactaatggccacgtattctgcagctttcctgcctgtggtcattggattggaccgccaggcagcagtactcaacccgcttggatcccgttcaggtgtaaggaaacttctgggggcagcctggggacttagtttcctgcttgccttcccccagctgttcctgttccacacggtccacnnngctggcccagtccctttcactcagtgtgtcaccaaaggcagcttcaaggctcaatggcaagagaccacctataacctcttcaccttctgctgcctcyttctgctgccactgactgccatggccatctgctatagccgcattgtcctcagtgtgtccaggccccagacaaggaaggggagccatgcccctgctggtgaatttgccctcccccgctcctttgacaattgtccccgtgttcgtctccgggccctgagactggccctgcttatcttgctgaccttcatcctctgctggacaccttattacctactgggtatgtggtactggttctcccccaccatgctaactgaagtccctcccagcctgagccacatccttttcctcttgggcctcctcaatgctcctttggatcctctcctctatggggccttcacccttggctgccgaagagggcaccaagaacttagtatagactcttctaaagaagggtctgggagaatgctccaagaggagattcatgcctttagacagctggaagtacaaaaaactgtgacatcaagaagggcaggagaaacaaaaggcatttctataacatctatctga

SEQ ID NO: 41 - protein, 376 aa, Homo sapiens
  misc_feature (70)..(70): The ′Xaa′ at location 70 stands for Cys, or Ser.
  misc_feature (209)..(209): The ′Xaa′ at location 209 stands for Leu, or Phe.
  misc_feature (789)..(791): codon 789-791 is the codon tga translated as selenocysteine (amino acid 166)
MSAGNGTPWAGEEVWAGSGVEVEGSELPTFSAAAKVRVGVTIVLFVSSAGGNLAVLWSVTRREPSQLRPXPVRRLFIHLAAADLLVTFVVMPLDATWNITVQWLAVDIACRTLMFLKLMATYSAAFLPVVIGLDRQAAVLNPLGSRSGVRKLLGAAWGLSFLLAFPQLFLFHTVHXAGPVPFTQCVTKGSFKAQWQETTYNLFTFCCLXLLPLTAMAICYSRIVLSVSRPQTRKGSHAPAGEFALPRSFDNCPRVRLRALRLALLILLTFILCWTPYYLLGMWYWFSPTMLTEVPPSLSHILFLLGLLNAPLDPLLYGAFTLGCRRGHQELSIDSSKEGSGRMLQEEIHAFRQLEVQKTVTSRRAGETKGISITSI

SEQ ID NO: 42 - DNA, 1364 bp, Homo sapiens
  CDS (264)..(1361)
  misc_feature (759)..(761): codon 759-761 is the codon tga translated as selenocysteine (amino acid 166)
cagtagaggcctgaagcccaggctggtctgtccaaggaaaaaggagcgtgattggtaccagatcttcgttccctgcagaaccttgacagttgaacaagtgacctcctccagaacagatggagagtctccagaagcagaggctttagtgaacgaaattcgcaataatcagctccagatcctgaaaaggagggcgaagaatcagtggccaaagctaaccgcttcatacccacacttcatcctcctcagtttctctccaggccaccatgtctgcaggcaacggcaccccttgggtggaggtggagggctcagagctgcccaccttctcggcagcagccaaggtccgagtgggagtgaccattgtgctgtttgtttcttcggctggagggaacctggcagtcctgtggtcagtgacacggcgggaacccagccagctccgcccctstccggtcaggagactcttcatccatttagcagccgccgacttactagtcacttttgtggttatgcccctagatgccacctggaatatcactgttcaatggctggctgtggacatcgcatgtcggacactgatgttcctgaaactaatggccacgtattctgcagctttcctgcctgtggtcattggattggaccgccaggcagcagtactcaacccgcttggatcccgttcaggtgtaaggaaacttctgggggcagcctggggacttagtttcctgcttgccttcccccagctgttcctgttccacacggtccacnnngctggcccagtccctttcactcagtgtgtcaccaaaggcagcttcaaggctcaatggcaagagaccacctataacctcttcaccttctgctgcctcyttctgctgccactgactgccatggccatctgctatagccgcattgtcctcagtgtgtccaggccccagacaaggaaggggagccatgcccctgctggtgaatttgccctcccccgctcctttgacaattgtccccgtgttcgtctccgggccctgagactggccctgcttatcttgctgaccttcatcctctgctggacaccttattacctactgggtatgtggtactggttctcccccaccatgctaactgaagtccctcccagcctgagccacatccttttcctcttgggcctcctcaatgctcctttggatcctctcctctatggggccttcacccttggctgccgaagagggcaccaagaacttagtatagactcttctaaagaagggtctgggagaatgctccaagaggagattcatgcctttagacagctggaagtacaaaaaactgtgacatcaagaagggcaggagaaacaaaaggcatttctataacatctatctga

SEQ ID NO: 43 - protein, 366 aa, Homo sapiens
  misc_feature (60)..(60): The ′Xaa′ at location 60 stands for Cys, or Ser.
  misc_feature (199)..(199): The ′Xaa′ at location 199 stands for Leu, or Phe.
  misc_feature (759)..(761): codon 759-761 is the codon tga translated as selenocysteine (amino acid 166)
MSAGNGTPWVEVEGSELPTFSAAAKVRVGVTIVLFVSSAGGNLAVLWSVTRREPSQLRPXPVRRLFIHLAAADLLVTFVVMPLDATWNITVQWLAVDIACRTLMFLKLMATYSAAFLPVVIGLDRQAAVLNPLGSRSGVRKLLGAAWGLSFLLAFPQLFLFHTVHXAGPVPFTQCVTKGSFKAQWQETTYNLFTFCCLXLLPLTAMAICYSRIVLSVSRPQTRKGSHAPAGEFALPRSFDNCPRVRLRALRLALLILLTFILCWTPYYLLGMWYWFSPTMLTEVPPSLSHILFLLGLLNAPLDPLLYGAFTLGCRRGHQELSIDSSKEGSGRMLQEEIHAFRQLEVQKTVTSRRAGETKGISITSI